(19) United States
(12) Patent Application Publication
(10) Pub. No.: US 2021/0371459 A1
(43) Pub. Date: Dec. 2, 2021

(54) DIMERIC PEPTIDE INHIBITORS OF APOPTOSIS PROTEINS

(71) Applicant: Hepagene Therapeutics (HK) Limited, Wan Chai (HK)

(72) Inventor: Xiaodong XU, Doylestown, PA (US)

(21) Appl. No.: 16/633,790

(22) PCT Filed: Jul. 24, 2018
(86) PCT No.: PCT/US2018/043550

§ 371 Date: Jan. 24, 2020

Related U.S. Application Data
(60) Provisional application No. 62/536,755, filed on Jul. 25, 2017.

(51) Int. Cl. C07K 5/062; C07K 5/083

(52) U.S. Cl. CPC C07K 5/06026; A61K 38/00; C07K 5/0806

(57) ABSTRACT

The present technology is directed to compounds, compositions, and methods related to treatment of cancers and viral infections mediated by IAPs. In particular the present compounds and compositions may be used to treat IAP-mediated ovarian cancer and hepatitis B infection.

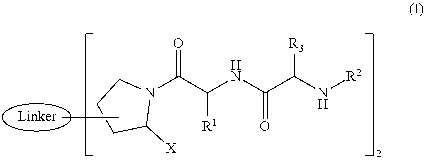

### CROSS-REFERENCE TO RELATED APPLICATIONS

This application claims the benefit of U.S. Provisional Application No. 62/536,755, filed Jul. 25, 2017, the content of which is incorporated herein by reference in its entirety.

### FIELD

The present technology is directed to compounds, compositions, and methods related to antagonizing inhibitor of apoptosis proteins (IAPs), including host cell IAPs (cIAPs). In particular, the present compounds and compositions may be used to treat various cancers, including, e.g., ovarian cancer and chronic hepatitis B infections.

### BACKGROUND

Apoptosis, also referred as programmed cell death, is a critical and highly regulated cell process that occurs in multicellular organisms, and apoptosis dysfunction is a hallmark of human cancers. Inhibitors of apoptosis proteins (IAPs), such as cellular inhibitor of apoptosis protein 1 and 2 (cIAP1 and cIAP2) and X-linked inhibitor of apoptosis protein (XIAP), have been identified as attractive targets for a new class of cancer therapy.

In 2015, Pellegrinia etc. (PNAS, 2015, 112(18), 5803-5808) demonstrated that the clinical-stage drug birinapant, which antagonizes host cell inhibitor of apoptosis proteins (cIAPs), promotes the killing of HBV-infected hepatocytes in a mouse model of HBV. Therefore, antagonists of cIAPs may also be efficacious in the treatment of chronic HBV infection and may promote elimination of virus.

### SUMMARY

In one aspect, the present technology provides a compound according to formula I, a stereoisomer thereof, or a pharmaceutically acceptable salt of the compound or the stereoisomer of the compound:

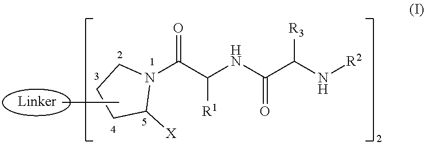

wherein
X is a bond to the Linker or, when the Linker is attached to positions, 2, 3, or 4 on the pyrrolidine ring (positions numbered as shown above), X is selected from

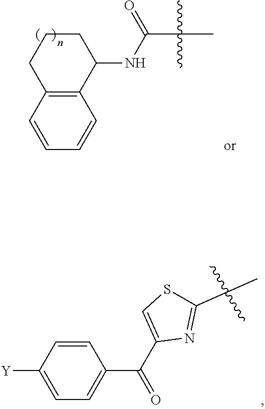

wherein Y is H or halogen;
R¹ and R³ are independently selected from a substituted or unsubstituted C₁₋₆ alkyl or a C₃₋₆ cycloalkyl group;
R² is H or a substituted or unsubstituted C₁₋₆ alkyl group;
m is 1, 2, 3, 4, 5, or 6;
n is 0, 1 or 2; and
Linker is selected from the group consisting of

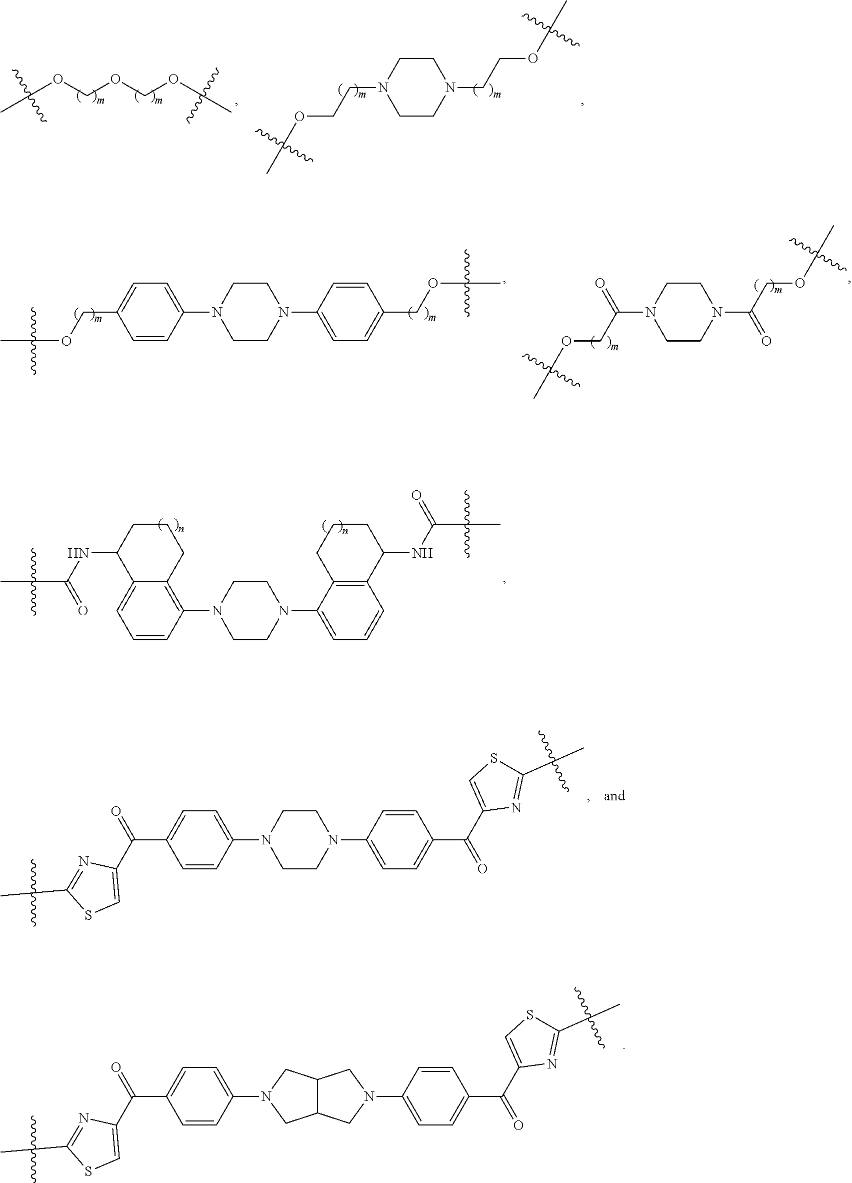

In a related aspect, a composition is provided that includes the compound of any one of the embodiments described herein and a pharmaceutically acceptable carrier.

In another aspect, a pharmaceutical composition is provided, the pharmaceutical composition including an effective amount of the compound of any one of the herein described embodiments for treating a IAP-mediated disorder or condition, such as various cancers (e.g., ovarian, fallopian tube, peritoneal cancers) or viral infections (e.g., chronic hepatitis B infection).

In another aspect, a method is provided that includes administering an effective amount of a compound of any one of the embodiments described herein, or administering a pharmaceutical composition including an effective amount of a compound of any one of the embodiments described herein, to a subject suffering from a cIAP-mediated disorder condition.

### DETAILED DESCRIPTION

In various aspects, the present technology provides compounds and methods for antagonizing the action of cIAP and the treatment of cIAP-mediated disorders and conditions. The compounds provided herein can be formulated into pharmaceutical compositions and medicaments that are useful in the disclosed methods. Also provided is the use of the compounds in preparing pharmaceutical formulations and medicaments.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated.

No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls, sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above.

In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above.

Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like.

Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R and R are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid).

When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, guanidines may exhibit the following isomeric forms in protic organic solution, also referred to as tautomers of each other:

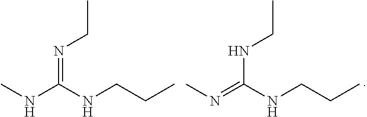

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

In one aspect, the present technology provides a compound of Formula I as described above. In some embodiments, the compound of Formula I is a compound of Formula IA:

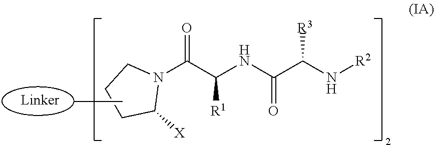

The variables Linker, X, R¹, R², and R³ may be defined as for the compounds of Formula I.

In some embodiments of compounds of Formula I or IA, Linker is

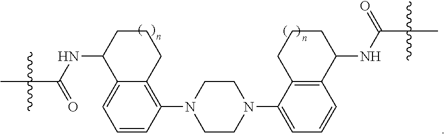

In some such embodiments, n is 0 or 1. In some embodiments, Linker is

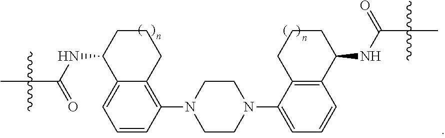

In some such embodiments, n is 0 or 1.

In some embodiments of compounds of Formula I or IA, Linker is

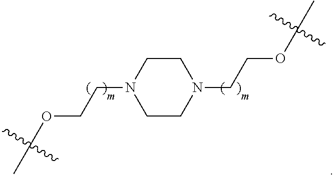

In some such embodiments, m may be 1, 2 or 3. For example, m may be 2.

In some embodiments, Linker is

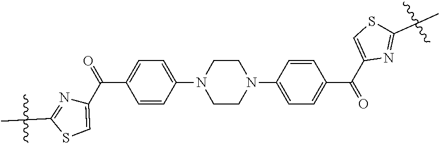 [SMILES: *c1nc(C(=O)c2ccc(N3CCN(c4ccc(C(=O)c5csc(*)n5)cc4)CC3)cc2)cs1]

In some embodiments, Linker is

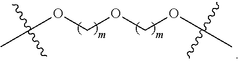

In some such embodiments, m may be 2 or 3. For example, m may be 2.

In some embodiments, Linker is

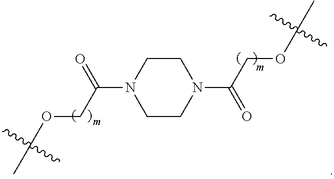

In some such embodiments, m may be 1, 2 or 3. For example, m may be 2.

In some embodiments, X is a bond to Linker. In certain embodiments, Linker is attached to the 3 position of the pyrrolidine of the compound of Formula I or IA. In any embodiment of compounds of Formula I or IA, m is 1, 2, or 3.

In any embodiments of compounds of Formula I or IA, X may be

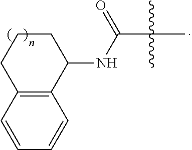

In any such embodiments, n may be 1.

In any embodiments of compounds of Formula I or IA, X may be

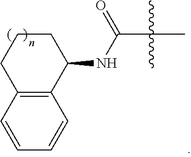

In any such embodiments, n may be 1.

In any embodiment, X may be

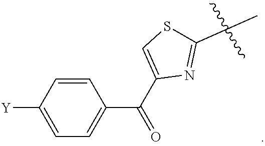

In any such embodiments, Y may be F.

In any embodiments of compounds of Formula I or IA, R¹ and R³ may be independently a methyl, ethyl, n-propyl, i-propyl, n-butyl, s-butyl, i-butyl, t-butyl, cyclopropyl, cyclobutyl, cyclohexyl, or cyclopentyl group. In any embodiments of compounds of Formula I or IA, R² may be a methyl, ethyl, n-propyl, i-propyl, n-butyl, i-butyl, or t-butyl group. In any embodiments R¹ may be cyclohexyl, and/or R² may methyl, and/or R³ may be methyl.

In an aspect of the present technology, a composition is provided that includes any one of the aspects and embodiments of compounds of formula I and a pharmaceutically acceptable carrier. In a related aspect, a pharmaceutical composition is provided which includes an effective amount of the compound of any one of the aspects and embodiments of compounds of formula I for treating an a cancer or a viral infection mediated by an IAP, e.g., a cIAP. The cancer or viral infection mediated by an IAP may be ovarian cancer, fallopian tube cancer, peritoneal cancer, and hepatitis B infection.

In another aspect, a method is provided that includes administering an effective amount of a compound of any one of the aspects and embodiments of compounds of formula I or administering a pharmaceutical composition comprising an effective amount of a compound of any one of the aspects and embodiments of compounds of formulas I to a subject suffering from a cancer or a viral infection mediated by an IAP, e.g., a cIAP. The cancer or viral infection mediated by an IAP may be ovarian cancer, fallopian tube cancer, peritoneal cancer, and hepatitis B infection.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of a cancer or a viral infection mediated by an IAP. The cancer or viral infection mediated by an IAP may be ovarian cancer, fallopian tube cancer, peritoneal cancer, and hepatitis B infection. Another example of an effective amount includes amounts or dosages that are capable of reducing symptoms associated with viral infection, such as, for example, virus titer and. As used herein, a “subject” or “patient” is a mammal, such as a cat, dog, rodent or primate. Typically the subject is a human, and, preferably, a human suffering from or suspected of suffering from an FXR-mediated or TGR5-mediated disorder or condition. The term “subject” and “patient” can be used interchangeably.

Thus, the instant present technology provides pharmaceutical compositions and medicaments comprising any of the compounds disclosed herein (e.g., compounds of formulas I) and a pharmaceutically acceptable carrier or one or more excipients or fillers. The compositions may be used in the methods and treatments described herein. Such compositions and medicaments include a therapeutically effective amount of any compound as described herein, including but not limited to a compound of formula I. The pharmaceutical composition may be packaged in unit dosage form.

The pharmaceutical compositions and medicaments may be prepared by mixing one or more compounds of the present technology, stereoisomers thereof, and/or pharmaceutically acceptable salts thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a cancers or viral infections associated with or mediated by IAPs, including but not limited to those described herein. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until for example, the desired therapeutic response is observed. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the symptoms of hyperlipidemia, such as, for example, a decrease in triglycerides in the blood stream. Effectiveness of the compositions and methods of the present technology may also be demonstrated by a decrease in the signs and symptoms of chronic liver disease, hypercholesteremia, obesity, metabolic syndrome, cardiovascular disease, gastrointestinal disease, atherosclerosis, renal disease, colorectal cancer, and stroke.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology.

The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### General Synthetic and Analytical Details

All reagents and materials are or were purchased from commercial vendors.

### Representative General Synthetic Schemes

The following compounds were or can be prepared as indicated in the following synthetic schemes using procedures known to those of ordinary skill in the art.

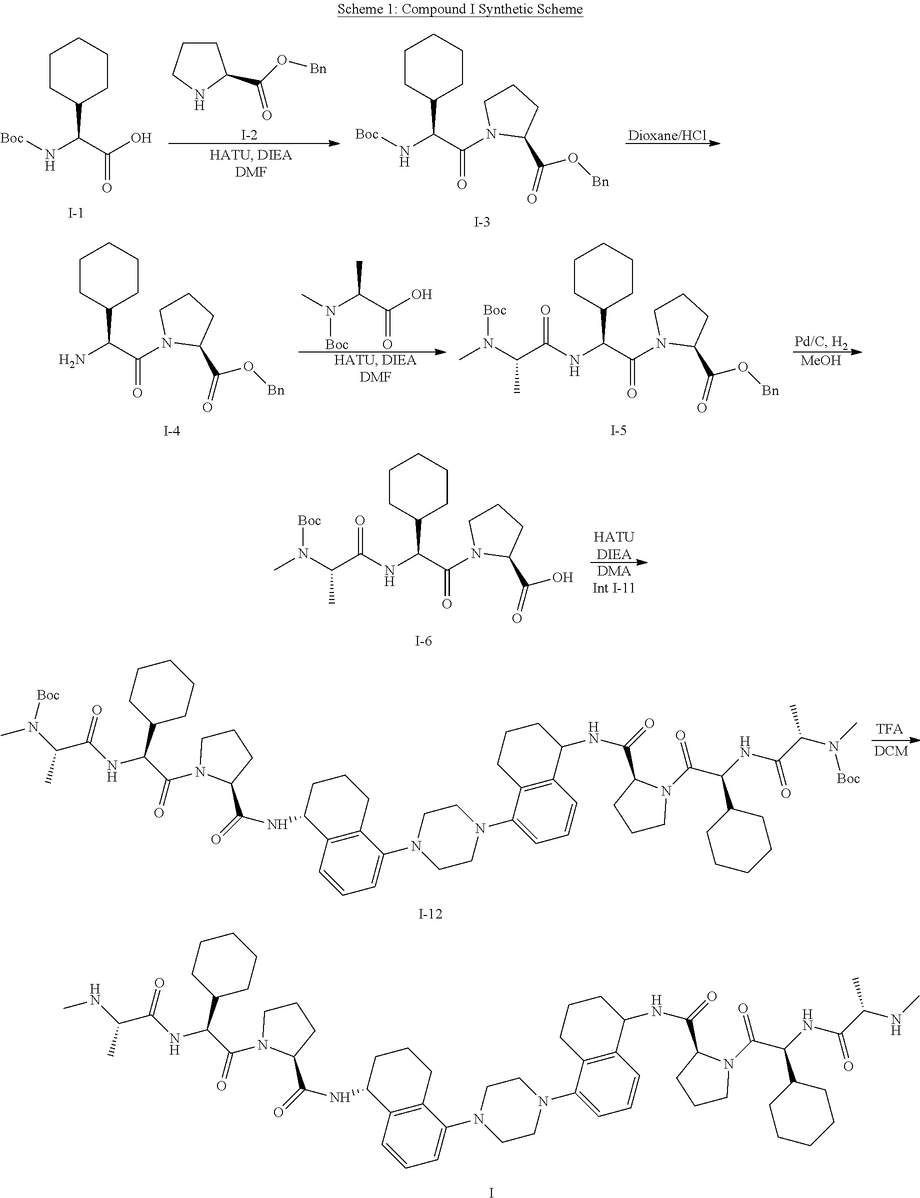

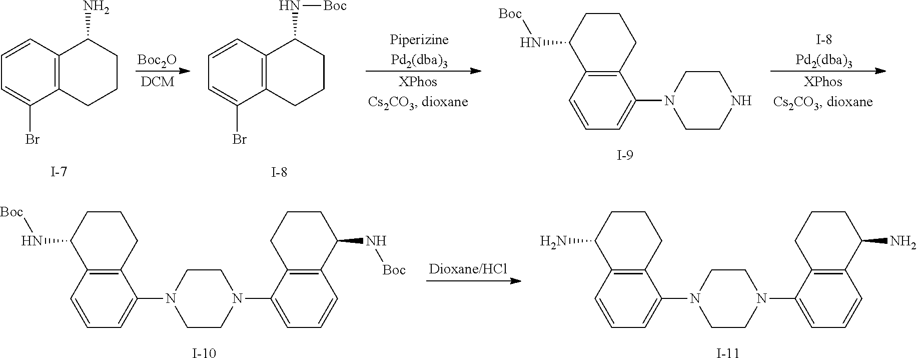

### Example 1: Synthesis of Compound I (Scheme 1)

Benzyl (2S)-1-[(2S)-2-[[(tert-butoxy)carbonyl]amino]-2-cyclohexylacetyl]pyrrolidine-2-carboxylate (Compound 1-3): To a solution of (2S)-2-[[(tert-butoxy)carbonyl]amino]-2-cyclohexylacetic acid (5 g, 19.43 mmol), DIEA (15 g, 116.06 mmol) and benzyl (2S)-pyrrolidine-2-carboxylate (9.4 g, 45.80 mmol) in DMF (100 mL) was added HATU (14.8 g, 38.92 mmol) batch-wise at room temperature. The resulting solution was stirred for 1 h at room temperature. The resulting mixture was diluted with 300 mL of EtOAc. The resulting mixture was washed successively with water and brine. The residue was concentrated under vacuum after dried over anhydrous sodium sulfate. The residue was applied onto a silica gel column with EtOAc/petroleum ether (1:3, v/v). This resulted in 7.9 g (91%) of the title compound as a colorless oil. LCMS (ESI, m/z): [M+H]⁺=445.3.

Benzyl (2S)-1-[(2S)-2-amino-2-cyclohexylacetyl]pyrrolidine-2-carboxylate (Compound 1-4): To a solution of Compound 1-3 (7.9 g, 17.77 mmol) in dioxane (50 mL) was added a solution of hydrogen chloride in dioxane (50 mL, 4M). The resulting solution was stirred for 6 h at room temperature. The residue was concentrated under vacuum. This resulted in 6.5 g of the title compound as a white solid. LCMS (ESI, m/z): [M+H]⁺=345.2.

Benzyl (2S)-1-[(2S)-2-[(2S)-2-[[(tert-butoxy)carbonyl](methyl)amino]propanamido]-2-cyclohexylacetyl]pyrrolidine-2-carboxylate (Compound 1-5): To a solution of (2S)-2-[[(tert-butoxy)carbonyl](methyl)amino]propanoic acid (3.2 g, 15.75 mmol), DIEA (6.1 g, 47.20 mmol) and Compound 1-4 (6.5 g, 18.87 mmol) in DMF (150 mL) was added HATU (7.2 g, 18.94 mmol) batch-wise at room temperature. The resulting mixture was stirred for 3 h at room temperature. The mixture was diluted with 250 mL of EtOAc. The mixture was washed successively with water and brine. The residue was concentrated under vacuum after dried over anhydrous sodium sulfate. The residue was applied onto a silica gel column with EtOAc/petroleum ether (1:3, v/v). This resulted in 8.0 g (96%) of the title compound as an orange oil. LCMS (ESI, m/z): [M+H]+=530.3.

(2S)-1-[(2S)-2-[(2S)-2-[[(tert-Butoxy)carbonyl](methyl)amino]propanamido]-2-cyclohexylacetyl]pyrrolidine-2-carboxylic acid (Compound 1-6): To a solution of Compound 1-5 (9.7 g, 18.31 mmol) in MeOH (150 mL) was added Pd/C (0.97 g). The resulting solution was stirred overnight at room temperature under H₂ atmosphere. The solids were filtered out. The filtrate was concentrated under vacuum. This resulted in 6.5 g (81%) of the title compound as a white solid. LCMS (ESI, m/z): [M+H]⁺=440.3.

tert-Butyl N-[(1R)-5-bromo-1,2,3,4-tetrahydronaphthalen-1-yl]carbamate (Compound 1-8): To a solution of (1R)-5-bromo-1,2,3,4-tetrahydronaphthalen-1-amine (3.1 g, 13.71 mmol) in DCM (20 mL) was added di-tert-butyl dicarbonate (3.16 g, 14.48 mmol). The resulting solution was stirred at room temperature for 6 h. The mixture was concentrated under vacuum. The residue was applied onto a silica gel column with EtOAc/petroleum ether (1:10, v/v). This resulted in 4.24 g (95%) of the title compound as a white solid. LCMS (ESI, m/z): [M+H]⁺=326.1.

tert-Butyl N-[(1R)-5-(piperazin-1-yl)-1,2,3,4-tetrahydronaphthalen-1-yl]carbamate (Compound 1-9): To a solution of Compound 1-8 (3 g, 9.21 mmol) in dioxane (50 mL) was added piperazine (3.18 g, 36.93 mmol), Pd₂(dba)₃ CHCl₃ (510 mg, 0.48 mmol), Xanphos (540 mg, 0.93 mmol) and Cs₂CO₃(8.7 g, 26.61 mmol). The resulting solution was stirred at 100° C. for overnight under N₂. The solids were filtered out. The resulting mixture was diluted with 50 mL of EA. The resulting mixture was washed successively with water and brine. The residue was concentrated under vacuum after dried over anhydrous sodium sulfate. The residue was applied onto a silica gel column with ACN/H₂O (1:1, v/v). This resulted in 1.16 mg (39%) of the title compound as a light yellow oil. LCMS (ESI, m/z): [M+H]⁺=332.2.

tert-Butyl N-[(1R)-5-[4-[(5R)-5-[[(tert-butoxy)carbonyl]amino]-5,6,7,8-tetrahydronaphthalen-1-yl]piperazin-1-yl]-1,2,3,4-tetrahydronaphthalen-1-yl]carbamate (Compound 1-10): To a solution of Compound 1-8 (1.14 g, 3.49 mmol) in dioxane (15 mL) was added Compound 1-9 (1.16 g, 3.50 mmol), Pd₂(dba)₃. CHCl₃ (190 mg, 0.18 mmol), X-Phos (330 mg, 0.69 mmol) and Cs₂CO₃ (2.86 g, 8.75 mmol). The resulting solution was stirred overnight at 100° C. under N₂ atmosphere. The solids were filtered out.

The resulting mixture was diluted with 15 mL of EA. The resulting mixture was washed successively with water and brine. The residue was concentrated under vacuum after dried over anhydrous sodium sulfate. The residue was applied onto a silica gel column with EtOAc/petroleum ether (1:6, v/v). This resulted in 1.3 g (66%) of the title compound as a yellow solid. LCMS (ESI, m/z): [M+H]⁺=577.4.

(1R)-5-[4-[(5R)-5-Amino-5,6,7,8-tetrahydronaphthalen-1-yl]piperazin-1-yl]-1,2,3,4-tetrahydronaphthalen-1-amine (Compound 1-11): To a solution of Compound 1-10 (1.3 g, 2.25 mmol) in dioxane (10 mL) was added a solution of hydrogen chloride in dioxane (10 mL, 4M). The resulting solution was stirred for 1 h at room temperature. The residue was concentrated under vacuum. This resulted in 840 mg (99%) of the title compound as a yellow solid. LCMS (ESI, m/z): [M+H]⁺=377.3

tert-Butyl N-[(1S)-1-[[(1S)-2-[(2S)-2-[[(1R)-5-[4-[(5R)-5-[(2S)-1-[(2S)-2-[(2S)-2-[[(tert-butoxy)carbonyl](methyl)amino]propanamido]-2-cyclohexylacetyl]pyrrolidine-2-amido]-5,6,7,8-tetrahydronaphthalen-1-yl]piperazin-1-yl]-1,2,3,4-tetrahydronaphthalen-1-yl]carbamoyl]pyrrolidin-1-yl]-1-cyclohexyl-2-oxoethyl]carbamoyl]ethyl]-N-methyl carbamate (Compound 1-12): To a solution of Compound 1-6 (1.75 g, 3.98 mmol), DIEA (1.03 g, 7.97 mmol) and Compound I-11 (500 mg, 1.33 mmol) in DMA (15 mL) was added HATU (1.52 g, 4.00 mmol) batch-wise at room temperature. The resulting mixture was stirred for 30 minutes at room temperature then quenched by adding 15 mL of water. The resulting mixture was extracted with 5×15 mL of EtOAc and the organic layers were combined. The resulting mixture was washed successively with water and brine. The residue was concentrated under vacuum after dried over anhydrous sodium sulfate. The crude product was applied onto a silica gel column with MeOH/DCM (99:1, v/v). This resulted in 963 mg (59%) of the title compound as a light yellow solid. LCMS (ESI, m/z): [M+H]⁺=1220.

(2S)-1-[(2S)-2-Cyclohexyl-2-[(2S)-2-(methylamino)propanamido]acetyl]-N-[(1R)-5-[4-[(5R)-5-[(2S)-1-[(2S)-2-cyclohexyl-2-[(2S)-2-(methylamino)propanamido]acetyl] pyrrolidine-2-amido]-5,6,7,8-tetrahydronaphthalen-1-yl]piperazin-1-yl]-1,2,3,4-tetrahydronaphthalen-1-yl]pyrrolidine-2-carboxamide (Compound I): To a solution of Compound 1-12 (963 mg, 0.79 mmol) in DCM (30 mL) was added TFA (3 mL). The resulting solution was stirred for 2 h at room temperature. The residue was concentrated under vacuum. The crude product was applied onto a reversed column with ethyl ACN/H₂O (7:3, v/v). This resulted in 372 mg (46%) of the title compound as a white solid. LCMS (ESI, m/z): [M+H]⁺=1019.8. ¹H NMR (400 MHz, CDCl₃, ppm): δ 7.61 (s, 2H), 7.15-7.07 (m, 4H), 6.98-6.95 (m, 4H), 5.14 (s, 2H), 4.63-4.51 (m, 4H), 3.85-3.81 (m, 2H), 3.63-3.53 (m, 2H), 3.10-2.95 (m, 10H), 2.86-2.82 (m, 2H), 2.72-2.66 (m, 2H), 2.57-2.48 (m, 2H), 2.35 (s, 6H), 2.16-2.03 (m, 6H), 1.93-1.76 (m, 8H), 1.66-1.57 (m, 12H), 1.28-1.24 (m, 8H), 1.13-0.88 (m, 10H).

Following the procedure described above for Scheme 1 and substituting the appropriate reagents, starting materials and purification methods known to those skilled in the art, the following compounds were prepared.

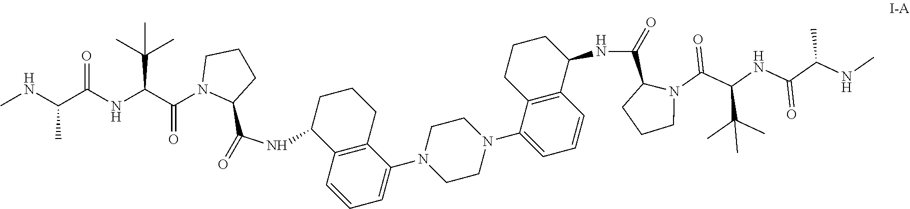

(2S,2′S,29S,34S)—N, N′-((1R,1′R)-5,5′-(Piperazine-1,4-diyl)bis(1,2,3,4-tetrahydronaphthalene-5,1-diyl))bis(1-((S)-3,3-dimethyl-2-((S)-2-(methylamino)propanamido)butanoyl)pyrrolidine-2-carboxamide) (Compound I-A): LCMS (ESI, m/z): [M+H]⁺=967.6. ¹H NMR (400 MHz, CDCl₃, ppm): δ 7.76 (m, 2H), 7.20-6.94 (m, 8H), 5.21-5.07 (m, 2H), 4.63-4.57 (m, 2H), 4.42-4.40 (m, 1H), 3.63-4.40 (m, 4H), 3.01-2.83 (m, 10H), 2.80-2.70 (m, 4H), 2.48-2.32 (m, 8H), 2.18-1.93 (m, 8H), 1.78-1.53 (m, 6H), 1.32-1.30 (m, 3H), 1.21-0.99 (m, 18H), 0.89-0.82 (m, 6H).

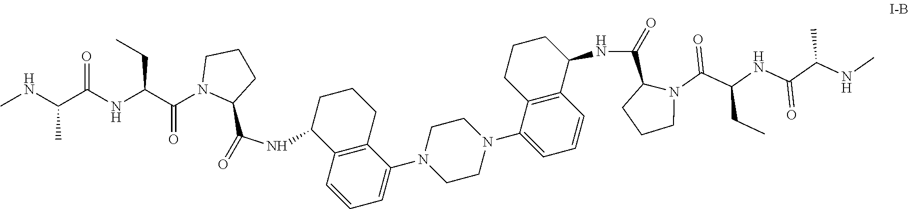

(2S)-1-[(2S)-2-[(2S)-2-(Methylamino)propanamido]butanoyl]-N-[(1R)-5-[4-[(5R)-5-[(2S)-1-[(2S)-2-[(2S)-2-(methylamino)propanamido]butanoyl]pyrrolidine-2-amido]-5,6,7,8-tetrahydronaphthalen-1-yl]piperazin-1-yl]-1,2,3,4-tetrahydronaphthalen-1-yl]pyrrolidine-2-carboxamide (Compound I-B): LCMS (ESI, m/z): [M+H]⁺=911.7.

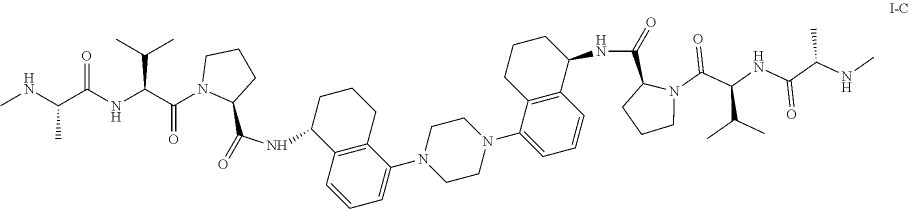

(S)-1-((S)-3-Methyl-2-((S)-2-(methylamino)propanamido)butanoyl)-N—((R)-5-(4-((R)-5-((S)-1-((S)-3-methyl-2-((S)-2-(methylamino)propanamido)butanoyl)pyrrolidine-2-carboxamido)-5,6,7,8-tetrahydronaphthalen-1-yl)piperazin-1-yl)-1,2,3,4-tetrahydronaphthalen-1-yl)pyrrolidine-2-carboxamide (Compound I-C): LCMS (ESI, m/z): [M+H]⁺=939.5.

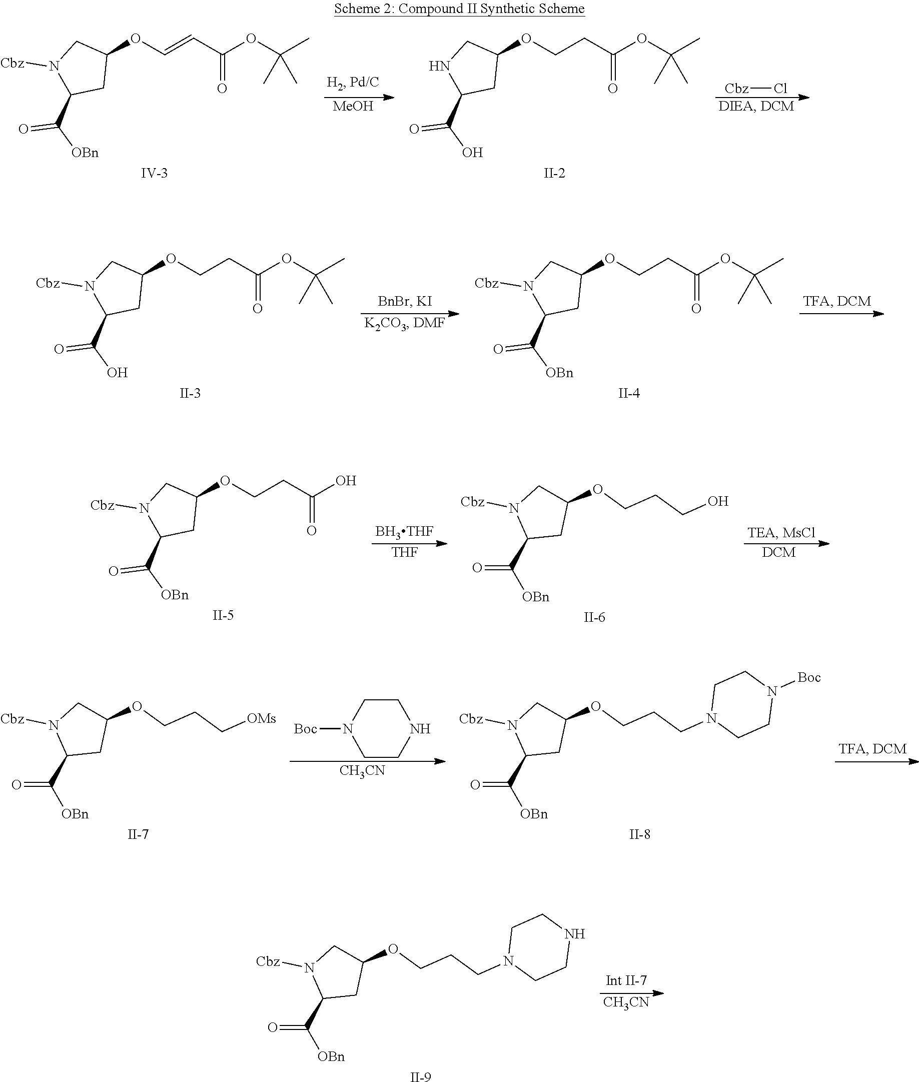

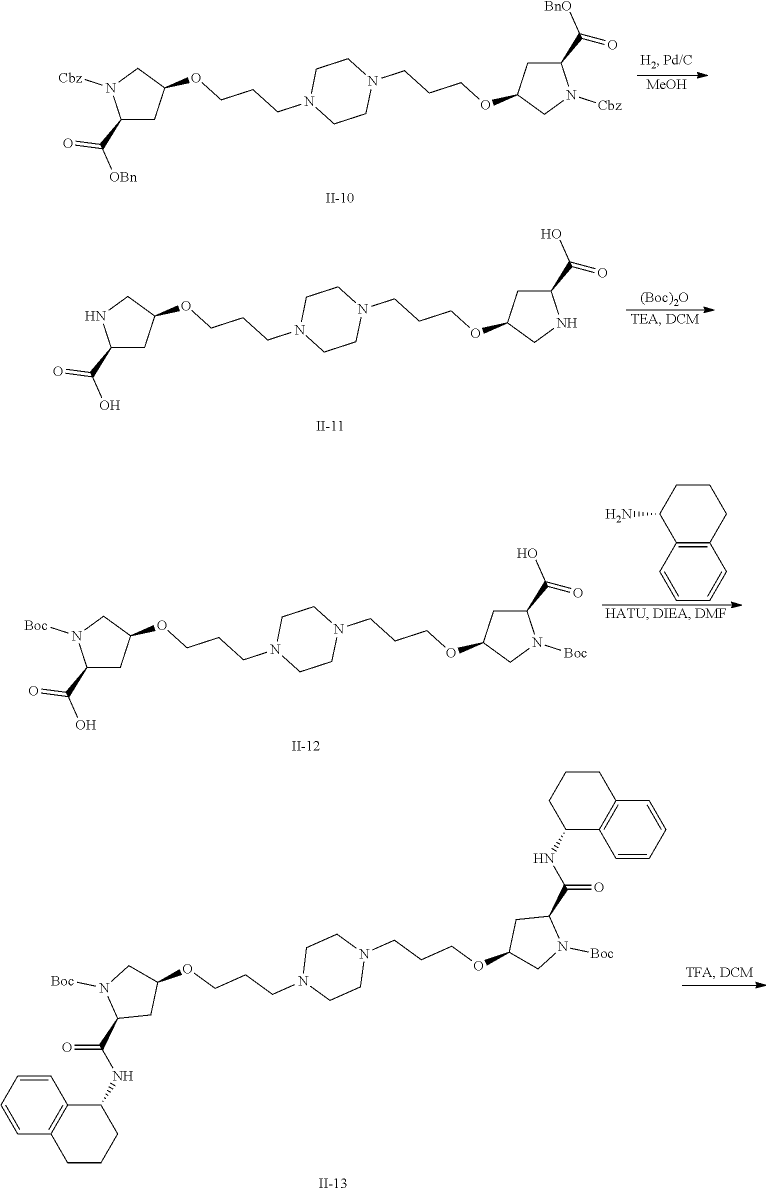

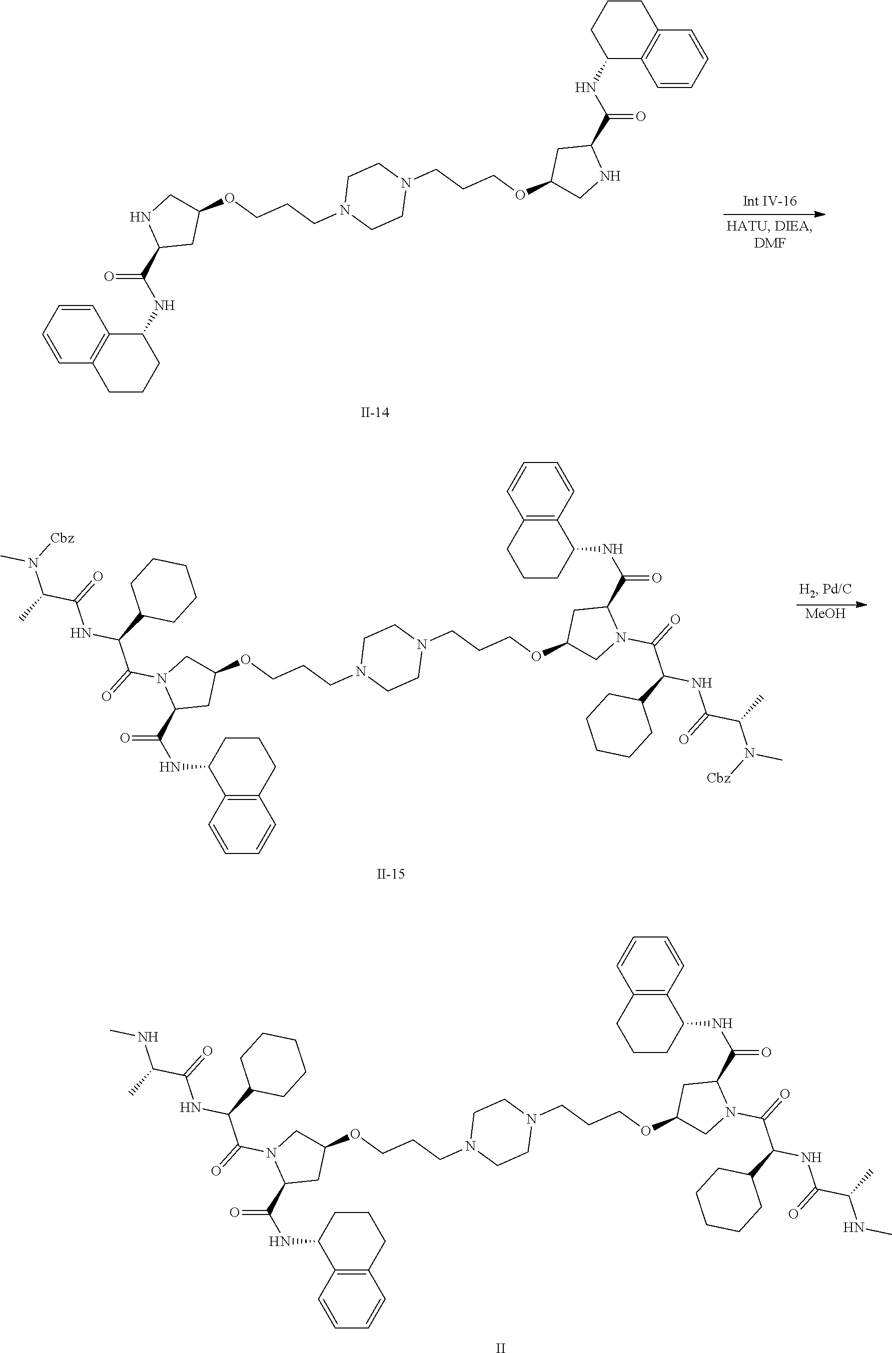

### Example 2: Synthesis of Compound II (Scheme 2)

(2S,4S)-4-[3-(tert-Butoxy)-3-oxopropoxy]pyrrolidine-2-carboxylic acid (Compound II-2): To a solution of Compound IV-3 (1.8 g, 3.86 mmol) in MeOH (25 mL) was added Pd/C (185 mg). The resulting mixture was stirred at room temperature for 3 h under H₂ atmosphere. After the reaction was completed, the mixture was filtered. The filtrate was evaporated in vacuo to afford the title compound (1 g, crude) as a yellow oil. LCMS (ESI, m/z): [M+H]⁺=260.1.

(2S,4S)-1-[(Benzyloxy)carbonyl]-4-[3-(tert-butoxy)-3-oxopropoxy]pyrrolidine-2-carboxylic acid (Compound II-3): To a solution of Compound II-2 (1.0 g, 3.86 mmol) in DCM (20 mL) was added DIEA (1.0 g, 7.74 mmol). The resulting mixture was stirred at room temperature for 30 min. Then a solution of benzyl carbonochloridate (990 mg, 5.80 mmol) in DCM (5 mL) was added dropwise to the mixture at 0° C. The resulting mixture was stirred at room temperature for 16 h. After the reaction was completed, the resulting mixture was concentrated to afford the title compound (1.5 g, crude) as a yellow solid. LCMS (ESI, m/z): [M+H]⁺=394.2.

(2S,4S)-Dibenzyl 4-(3-tert-butoxy-3-oxopropoxy)pyrrolidine-1,2-dicarboxylate (Compound II-4): To a solution of Compound II-3 (1.5 g, 3.81 mmol) in DMF (25 mL) was added K₂CO₃ (1.2 g, 8.39 mmol), KI (63 mg, 0.38 mmol) and (bromomethyl)benzene (1.9 g, 11.40 mmol) at room temperature. The resulting mixture was stirred at room temperature for 16 h. The mixture was diluted with H₂O and extracted with EtOAc. The organic layer was washed with brine, dried over anhydrous sodium sulfate and filtered. The filtrate was concentrated under vacuum. The residue was purified by flash column chromatography with EtOAc/petroleum ether (1:1, v/v) to afford the title compound (1.2 g, 65%) as a yellow oil. LCMS (ESI, m/z): [M+H]⁺=484.2.

3-[[(3S,5S)-1,5-bis[(Benzyloxy)carbonyl]pyrrolidin-3-yl]oxy]propanoic acid (Compound II-5): To a solution of Compound II-4 (1.2 g, 2.48 mmol) in DCM (50 mL) was added TFA (5 mL). The resulting mixture was stirred at room temperature for 2 h. The mixture was concentrated under vacuum to afford the title compound (1.02 g, crude) as a yellow oil. LCMS (ESI, m/z): [M+H]⁺=428.2.

(2S,4S)-Dibenzyl 4-(3-hydroxypropoxy)pyrrolidine-1,2-dicarboxylate (Compound II-6): To a solution of Compound II-5 (1.0 g, 2.38 mmol) in THF (30 mL) was added BH₃.THF (12 mL) dropwise at 0° C. under N₂ atmosphere. The resulting mixture was stirred at room temperature for 16 h under N₂ atmosphere. The mixture was concentrated under vacuum. The residue was purified by flash column chromatography with DCM/EtOAc (1:1, v/v) to afford the title compound (880 mg, 89%) as a colorless oil. LCMS (ESI, m/z): [M+H]⁺=414.2.

(2S,4S)-Dibenzyl 4-(3-(methylsulfonyloxy)propoxy)pyrrolidine-1,2-dicarboxylate (Compound II-7): To a solution of Compound II-6 (880 mg, 2.13 mmol) in DCM (10 mL) was added TEA (237 mg, 2.34 mmol). The resulting mixture was stirred at room temperature for 30 min. Then methanesulfonyl chloride (268 mg, 2.34 mmol) was added dropwise to the mixture at 0° C. The resulting mixture was stirred at room temperature for 5 h. The reaction mixture was diluted with H₂O and extracted with DCM. The organic layer was washed with brine, dried over anhydrous sodium sulfate and filtered. The filtrate was concentrated under vacuum to afford the title compound (1.07 g, crude) as a yellow oil. LCMS (ESI, m/z): [M+H]⁺=492.2.

(2S,4S)-Dibenzyl 4-(3-(4-(tert-butoxycarbonyl)piperazin-1-yl)propoxy)pyrrolidine-1,2-dicarboxylate (Compound II-8): To a solution of Compound II-7 (1.1 g, 2.34 mmol) in CH₃CN (10 mL) was added tert-butyl piperazine-1-carboxylate (1.2 g, 6.56 mmol). The resulting mixture was stirred at 60° C. for 16 h. The reaction mixture was concentrated under vacuum. The residue was purified by flash column chromatography with DCM/MeOH (13:1, v/v) to afford the title compound (1.2 g, 99%) as an orange oil.

LCMS (ESI, m/z): [M+H]⁺=582.3.

(2S,4S)-Dibenzyl 4-(3-(piperazin-1-yl)propoxy)pyrrolidine-1,2-dicarboxylate (Compound II-9): To a solution of Compound II-8 (1.2 g, 2.15 mmol) in DCM (20 mL) was added TFA (5 mL). The resulting mixture was stirred at room temperature for 2 h. The reaction mixture was concentrated under vacuum to afford the title compound (970 mg, crude) as an orange oil. LCMS (ESI, m/z): [M+H]⁺=482.3.

(2S,2'S,4S,4'S)-Tetrabenzyl 4,4′-(3,3′-(piperazine-1,4-diyl)bis(propane-3,1-diyl))bis(oxy)dipyrrolidine-1,2-dicarboxylate (Compound 11-10): To a solution of Compound II-9 (970 mg, 2.01 mmol) in CH₃CN (7 mL) was added Compound II-7 (825 mg, 1.68 mmol). The resulting mixture was stirred at 60° C. for 48 h. The reaction mixture was concentrated under vacuum. The residue was purified by flash column chromatography with DCM/MeOH (9:1, v/v) to afford the title compound (824 mg, 56%) as an orange oil. LCMS (ESI, m/z): [M+H]⁺=877.4.

(2S,4S)-4-[3-[4-(3-[[(3S,5S)-5-Carboxypyrrolidin-3-yl]oxy]propyl)piperazin-1-yl]propoxy]pyrrolidine-2-carboxylic acid (Compound 11-11): To a solution of Compound II-10 (824 mg, 0.94 mmol) in MeOH (10 mL) was added Pd/C (100 mg). The resulting mixture was stirred at room temperature for 16 h under H₂ atmosphere. After the reaction was completed, the mixture was filtered. The filtrate was evaporated in vacuo to afford the title compound (400 mg, crude) as a yellow oil. LCMS (ESI, m/z): [M+H]⁺=429.3.

(2S,4S)-1-[(tert-Butoxy)carbonyl]-4-[3-[4-(3-[[(3S,5S)-1-[(tert-butoxy)carbonyl]-5-carboxypyrrolidin-3-yl] oxy] propyl)piperazin-1-yl]propoxy]pyrrolidine-2-carboxylic acid (Compound 11-12): To a solution of Compound II-11 (400 mg, 0.93 mmol) in DCM (7 mL) was added TEA (207.8 mg, 2.05 mmol). The resulting mixture was stirred at room temperature for 30 min. Then a solution of di-tert-butyl dicarbonate (448.2 mg, 2.05 mmol) in DCM was added dropwise to the mixture at 0° C. The resulting mixture was stirred at room temperature for 3 h. The mixture was diluted with H₂O and extracted with DCM. The organic layer was washed with brine, dried over anhydrous sodium sulfate and filtered. The filtrate was purified by flash column chromatography with DCM/MeOH (10:1, v/v) to afford the title compound (220 mg, 37%) as a yellow oil. LCMS (ESI, m/z): [M+H]⁺=629.4.

tert-Butyl (2S,4S)-4-[3-[4-(3-[[(3S,5S)-1-[(tert-butoxy)carbonyl]-5-[[(1R)-1,2,3,4-tetrahydronaphthalen-1-yl]carbamoyl]pyrrolidin-3-yl]oxy]propyl)piperazin-1-yl] propoxy]-2-[[(1R)-1,2,3,4-tetrahydronaphthalen-1-yl] carbamoyl] pyrrolidine-1-carboxylate (Compound 11-13): To a solution of Compound 11-12 (220 mg, 0.35 mmol) in DMF (7 mL) was added HATU (399.1 mg, 1.05 mmol), DIEA (271.3 mg, 2.10 mmol) and (1R)-1,2,3,4-tetrahydronaphthalen-1-amine (154.5 mg, 1.05 mmol) at room temperature. The resulting mixture was stirred at room temperature for 3 h. The reaction mixture was diluted with H₂O and extracted with EtOAc. The organic layer was washed with brine, dried over anhydrous sodium sulfate and filtered. The filtrate was concentrated under vacuum. The residue was purified by flash column chromatography with DCM/MeOH (12:1, v/v) to afford the title compound (280 mg, 90%) as a yellow oil. LCMS (ESI, m/z): [M+H]⁺=887.6.

(2S,4S)—N-[(1R)-1,2,3,4-Tetrahydronaphthalen-1-yl]-4-[3-[4-(3-[[(3S,5S)-5-[[(1R)-1,2,3,4-tetrahydronaphthalen-1-yl]carbamoyl]pyrrolidin-3-yl]oxy]propyl)piperazin-1-yl]propoxy]pyrrolidine-2-carboxamide (Compound 11-14): To a solution of Compound 11-13 (280 mg, 0.31 mmol) in DCM (5 mL) was added TFA (1 mL). The resulting mixture was stirred at room temperature for 2 h. The mixture was concentrated under vacuum to afford the title compound (125 mg, crude) as a yellow oil. LCMS (ESI, m/z): [M+H]⁺=687.5.

Benzyl N-[(1S)-1-[[(1S)-2-[(2S,4S)-4-[3-[4-(3-[[(3S,5S)-1-[(2S)-2-[(2S)-2-[[(benzyloxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]oxy]propyl)piperazin-1-yl]propoxy]-2-[[(1R)-1,2,3,4-tetrahydronaphthalen-1-yl]carbamoyl]pyrrolidin-1-yl]-1-cyclohexyl-2-oxoethyl]carbamoyl]ethyl]-N-methylcarbamate (Compound 11-15): To a solution of Compound IV-16 (205.5 mg, 0.55 mmol) in DMF (5 mL) was added HATU (207.6 mg, 0.55 mmol), DIEA (141.1 mg, 1.09 mmol) and Compound 11-14 (125 mg, 0.18 mmol) at room temperature. The resulting mixture was stirred at room temperature for 3 h.

The mixture was diluted with H₂O and extracted with EtOAc. The organic layer was washed with brine, dried over anhydrous sodium sulfate and filtered. The filtrate was concentrated under vacuum. The residue was purified by flash column chromatography with MeOH/DCM (1:13, v/v) to afford the title compound (218 mg, 85%) as a white solid. LCMS (ESI, m/z): [M+H]⁺=1404.1.

(2S,4S)-1-[(2S)-2-Cyclohexyl-2-[(2S)-2-(methylamino)propanamido]acetyl]-4-[3-[4-(3-[[(3S,5S)-1-[(2S)-2-cyclohexyl-2-[(2S)-2-(methylamino)propanamido]acetyl]-5-[[(1R)-1,2,3,4-tetrahydronaphthalen-1-yl]carbamoyl]pyrrolidin-3-yl] oxy] propyl)piperazin-1-yl] propoxy]-N-[(1R)-1,2,3,4-tetrahydronaphthalen-1-yl]pyrrolidine-2-carboxamide (Compound II): To a solution of Compound 11-15 (218 mg, 0.16 mmol) in MeOH (8 mL) was added Pd/C (20 mg). The resulting mixture was stirred at room temperature for 5 h under H₂ atmosphere. After the reaction was completed, the mixture was filtered. The filtrate was purified by Prep-HPLC with the following conditions: Column: XSelect CSH Prep C18 OBD Column, 19×250 mm, 5 um; Mobile Phase A: Water (0.05% TFA), Mobile Phase B: ACN; Flow rate: 25 mL/min; Gradient: 10% B to 42% B in 7 min; 254/220 nm; Rt: 7 min to afford the title compound (20 mg, 10%) as a white solid. LCMS (ESI, m/z): [M+H]⁺=1135.8. ¹H NMR (400 MHz, CD₃OD-d₄, ppm): δ 7.35-7.25 (m, 2H), 7.14-7.09 (m, 6H), 5.10-4.90 (m, 3H), 4.52-4.46 (m, 3H), 4.21-4.02 (m, 3H), 3.90-3.86 (m, 4H), 3.80-3.40 (m, 7H), 3.31-2.88 (m, 7H), 2.81-2.75 (m, 6H), 2.66-2.59 (m, 6H), 2.45-2.02 (m, 5H), 1.99-1.61 (m, 24H), 1.53-1.50 (m, 4H), 1.47 (d, J=6.8 Hz, 2H), 1.25-1.13 (m, 10H).

Following the procedure described above for Scheme 2 and substituting the appropriate reagents, starting materials and purification methods known to those skilled in the art, the following compounds were prepared.

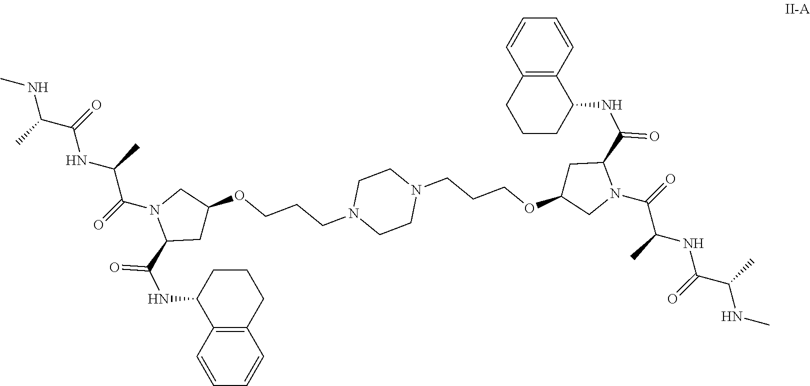

(2S,4S)-1-[(2S)-2-[(2S)-2-(Methylamino)propanamido] propanoyl]-4-{3-[4-(3-{[(3S,5S)-1-[(2S)-2-[(2S)-2-(methylamino)propanamido]propanoyl]-5-{[(1R)-1,2,3,4-tetrahydronaphthalen-1-yl]carbamoyl}pyrrolidin-3-yl]oxy}propyl)piperazin-1-yl]propoxy}-N-[(1R)-1,2,3,4-tetrahydronaphthalen-1-yl]pyrrolidine-2-carboxamide (Compound II-A):

LCMS (ESI, m/z): [M+H]⁺=1000.3. ¹H NMR (300 MHz, DMSO-d₆, ppm): δ 8.25-7.91 (m, 2H), 7.85-7.82 (m, 2H), 7.21-7.04 (m, 8H), 4.95-4.80 (m, 2H), 4.61-4.52 (m, 2H), 4.28-4.20 (m, 2H), 4.13-3.89 (m, 4H), 3.74-3.17 (m, 6H), 2.98-2.83 (m, 2H), 2.80-2.58 (m, 4H), 2.47-1.93 (m, 20H), 1.91-1.49 (m, 14H), 1.25-1.14 (m, 6H), 1.05 (d, J=6.6 Hz, 6H).

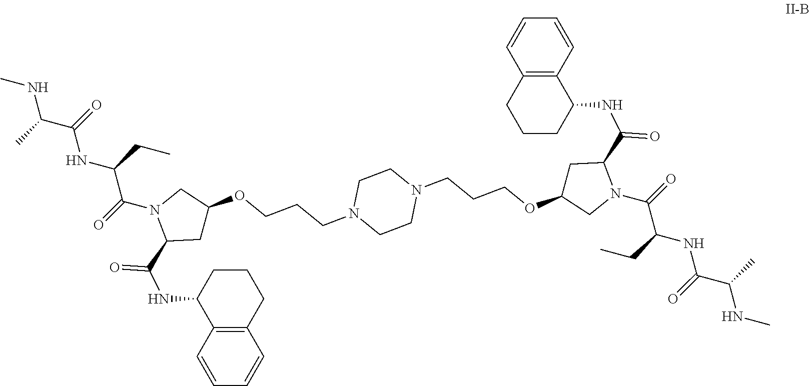

(2S,4S)-1-[(2S)-2-[(2S)-2-(Methylamino)propanamido] butanoyl]-4-{3-[4-(3-{[(3S,5S)-1-[(2S)-2-[(2S)-2-(methylamino)propanamido]butanoyl]-5-{[(1R)-1,2,3,4-tetrahydronaphthalen-1-yl]carbamoyl}pyrrolidin-3-yl]oxy}propyl)piperazin-1-yl]propoxy}-N-[(1R)-1,2,3,4-tetrahydronaphthalen-1-yl]pyrrolidine-2-carboxamide (Compound II-B):

LCMS (ESI, m/z): [M+H]⁺=1027.7. ¹H NMR (300 MHz, DMSO-d₆, ppm): δ 8.29-7.83 (m, 4H), 7.36-7.19 (m, 2H), 7.18-7.02 (m, 6H), 5.00-4.83 (m, 2H), 4.55-4.23 (m, 4H), 4.14-3.94 (m, 4H), 3.47-3.37 (m, 6H), 3.00-2.83 (m, 2H), 2.80-2.61 (m, 4H), 2.41-2.10 (m, 20H), 1.91-1.40 (m, 18H), 1.06 (d, J=6.9 Hz, 6H), 0.86-0.81 (m, 6H).

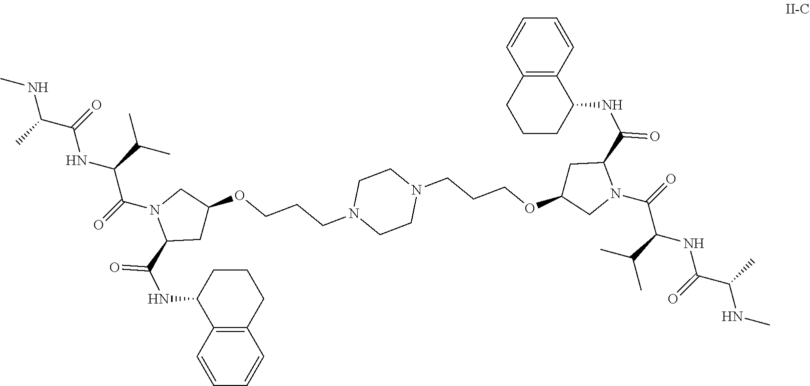

(2S,4S)-1-[(2S)-3-Methyl-2-[(2S)-2-(methylamino)propanamido] butanoyl]-4-{3-[4-(3-{[(3S,5S)-1-[(2S)-3-methyl-2-[(2S)-2-(methylamino)propanamido]butanoyl]-5-{[(1R)-1,2,3,4-tetrahydronaphthalen-1-yl]carbamoyl}pyrrolidin-3-yl]oxy}propyl)piperazin-1-yl]propoxy}-N-[(1R)-1,2,3,4-tetrahydronaphthalen-1-yl]pyrrolidine-2-carboxamide (Compound II-C):

LCMS (ESI, m/z): [M+H]⁺=1055.6. ¹H NMR (300 MHz, CD₃OD-d₄, ppm): δ 7.48-7.28 (m, 2H), 7.21-7.04 (m, 6H), 5.10-5.02 (m, 2H), 4.55-4.41 (m, 4H), 4.22-4.11 (m, 4H), 3.70-3.59 (m, 2H), 3.59-3.38 (m, 5H), 3.22-3.09 (m, 2H), 2.85-2.72 (m, 4H), 2.54-2.35 (m, 13H), 2.31 (s, 6H), 2.24-2.05 (m, 4H), 2.04-1.65 (m, 12H), 1.29-1.17 (m, 6H), 1.11-0.95 (m, 12H).

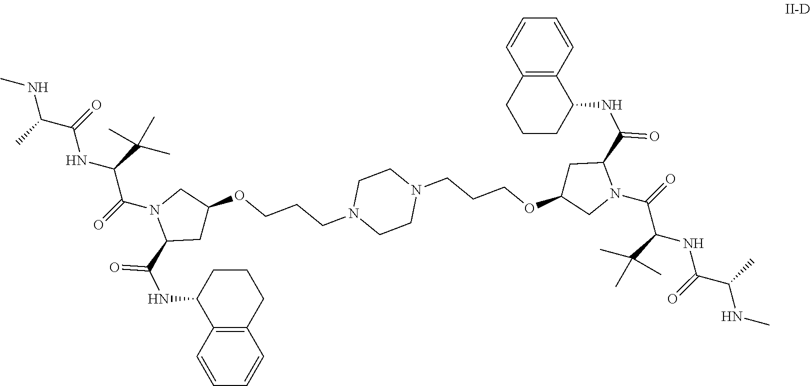

(2S,4S)-1-[(2S)-3,3-Dimethyl-2-[(2S)-2-(methylamino)propanamido] butanoyl]-4-[3-[4-(3-[[(3S,5S)-1-[(2S)-3,3-dimethyl-2-[(2S)-2-(methylamino)propanamido]butanoyl]-5-[[(1R)-1,2,3,4-tetrahydronaphthalen-1-yl] carbamoyl] pyrrolidin-3-yl] oxy]propyl)piperazin-1-yl] propoxy]-N-[(1R)-1,2,3,4-tetrahydronaphthalen-1-yl]pyrrolidine-2-carboxamide (Compound II-D):

LCMS (ESI, m/z): [M+H]⁺=1084.4. ¹H NMR (300 MHz, DMSO-d₆, ppm): δ 8.09-8.00 (m, 1H), 7.89-7.81 (m, 2H), 7.51-7.47 (m, 1H), 7.38-7.21 (m, 2H), 7.20-7.01 (m, 6H), 5.02-4.79 (m, 2H), 4.49-4.46 (m, 1H), 4.39-4.28 (m, 3H), 4.15-4.02 (m, 3H), 4.01-3.89 (m, 1H), 3.69-3.59 (m, 1H), 3.49-3.35 (m, 5H), 3.02-2.90 (m, 2H), 2.79-2.65 (m, 4H), 2.48-2.20 (m, 13H), 2.19-2.02 (m, 9H), 1.93-1.48 (m, 14H), 1.15-0.89 (m, 24H).

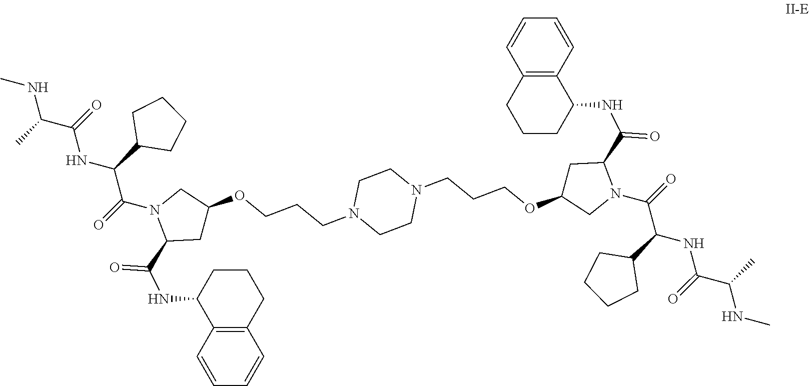

(S,S,2S,2'S,4S,4'S)-4,4′-((Piperazine-1,4-diylbis(propane-3,1-diyl))bis(oxy))bis(l-((S)-2-cyclopentyl-2-((S)-2-(methylamino)propanamido)acetyl)-N—((R)-1,2,3,4-tetrahydronaphthalen-1-yl)pyrrolidine-2-carboxamide) (Compound II-E): LCMS (ESI, m/z): [M+H]⁺=1107.6.

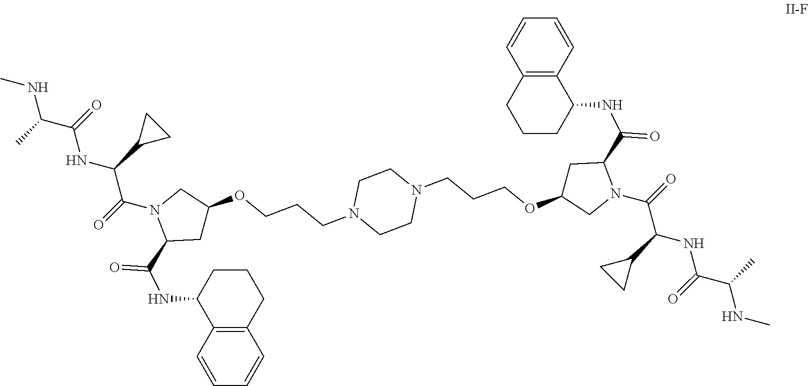

(2S,4S)-1-[(2S)-2-Cyclopropyl-2-[(2S)-2-(methylamino)propanamido]acetyl]-4-[3-[4-(3-[[(3S,5S)-1-[(2S)-2-cyclopropyl-2-[(2S)-2-(methylamino)propanamido]acetyl]-5-[[(1R)-1,2,3,4-tetrahydronaphthalen-1-yl]carbamoyl]pyrrolidin-3-yl] oxy] propyl)piperazin-1-yl] propoxy]-N-[(1R)-1,2,3,4-tetrahydronaphthalen-1-yl]pyrrolidine-2-carboxamide (Compound II-F):

LCMS (ESI, m/z): [M+H]⁺=1051.7. ¹H NMR (300 MHz, DMSO-d₆; ppm): δ 8.25-7.99 (m, 2H), 7.88-7.60 (m, 2H), 7.34-7.03 (m, 8H), 4.99-4.83 (m, 2H), 4.52-4.21 (m, 4H), 4.11-3.65 (m, 4H), 3.49-3.31 (m, 6H), 2.98-2.81 (m, 2H), 2.79-2.66 (m, 4H), 2.45-2.11 (m, 19H), 2.05-1.89 (m, 3H), 1.87-1.75 (m, 4H), 1.74-1.48 (m, 8H), 1.20-1.02 (m, 8H), 0.49-0.19 (m, 8H).

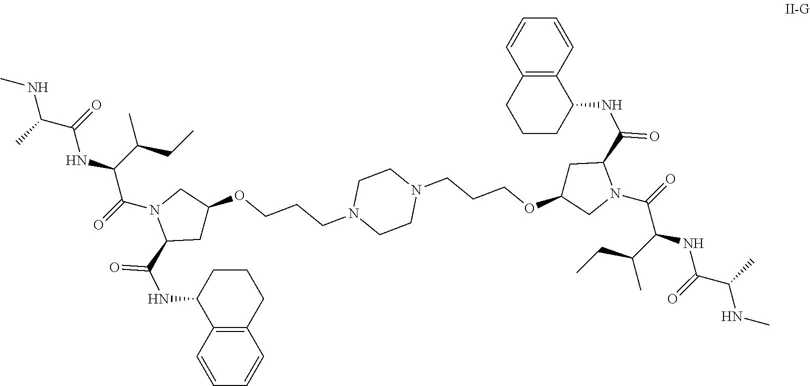

(2S,4S)-1-[(2S)-3-Methyl-2-[(2S)-2-(methylamino)propanamido] pentanoyl]-4-{3-[4-(3-{[(3S,5S)-1-[(2S)-3-methyl-2-[(2S)-2-(methylamino)propanamido]pentanoyl]-5-{[(1R)-1,2,3,4-tetrahydronaphthalen-1-yl]carbamoyl}pyrrolidin-3-yl]oxy}propyl)piperazin-1-yl]propoxy}-N-[(1R)-1,2,3,4-tetrahydronaphthalen-1-yl]pyrrolidine-2-carboxamide (Compound II-G):

LCMS (ESI, m/z): [M+H]⁺=1083.7. ¹H NMR (400 MHz, DMSO-d₆; ppm): δ 8.10-7.39 (m, 4H), 7.32-6.93 (m, 8H), 4.98-4.85 (m, 2H), 4.51-4.38 (m, 2H), 4.37-4.21 (m, 2H), 4.20-3.58 (m, 5H), 3.48-3.39 (m, 4H), 3.29-3.24 (m, 1H), 3.00-2.88 (m, 2H), 2.79-2.65 (m, 4H), 2.48-2.02 (m, 21H), 1.90-1.25 (m, 19H), 1.19-0.98 (m, 8H), 1.95-1.71 (m, 12H).

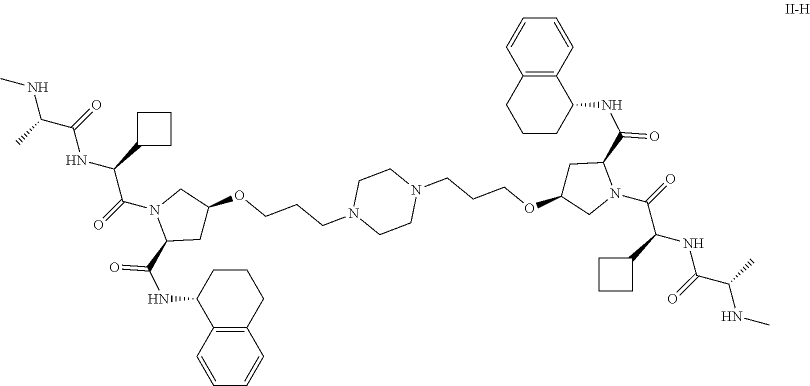

(2S,4S)-1-[(2S)-2-Cyclobutyl-2-[(2S)-2-(methylamino)propanamido]acetyl]-4-[3-[4-(3-[[(3S,5S)-1-[(2S)-2-cyclobutyl-2-[(2S)-2-(methylamino)propanamido]acetyl]-5-[[(1R)-1,2,3,4-tetrahydronaphthalen-1-yl]carbamoyl]pyrrolidin-3-yl] oxy] propyl)piperazin-1-yl] propoxy]-N-[(1R)-1,2,3,4-tetrahydronaphthalen-1-yl]pyrrolidine-2-carboxamide (Compound II-H):

LCMS (ESI, m/z): [M+H]⁺=1080.4. ¹H NMR (300 MHz, DMSO-d₆) δ 8.21-7.95 (m, 2H), 7.94-7.61 (m, 2H), 7.36-7.19 (m, 2H), 7.18-7.00 (m, 6H), 4.98-4.81 (m, 2H), 4.72-4.55 (m, 2H), 4.51-4.38 (m, 1H), 4.32-4.18 (m, 2H), 4.09-3.89 (m, 4H), 3.49-3.35 (m, 5H), 3.01-2.88 (m, 2H), 2.78-2.55 (m, 6H), 2.40-2.11 (m, 20H), 1.99-1.49 (m, 28H), 1.18-1.02 (m, 6H).

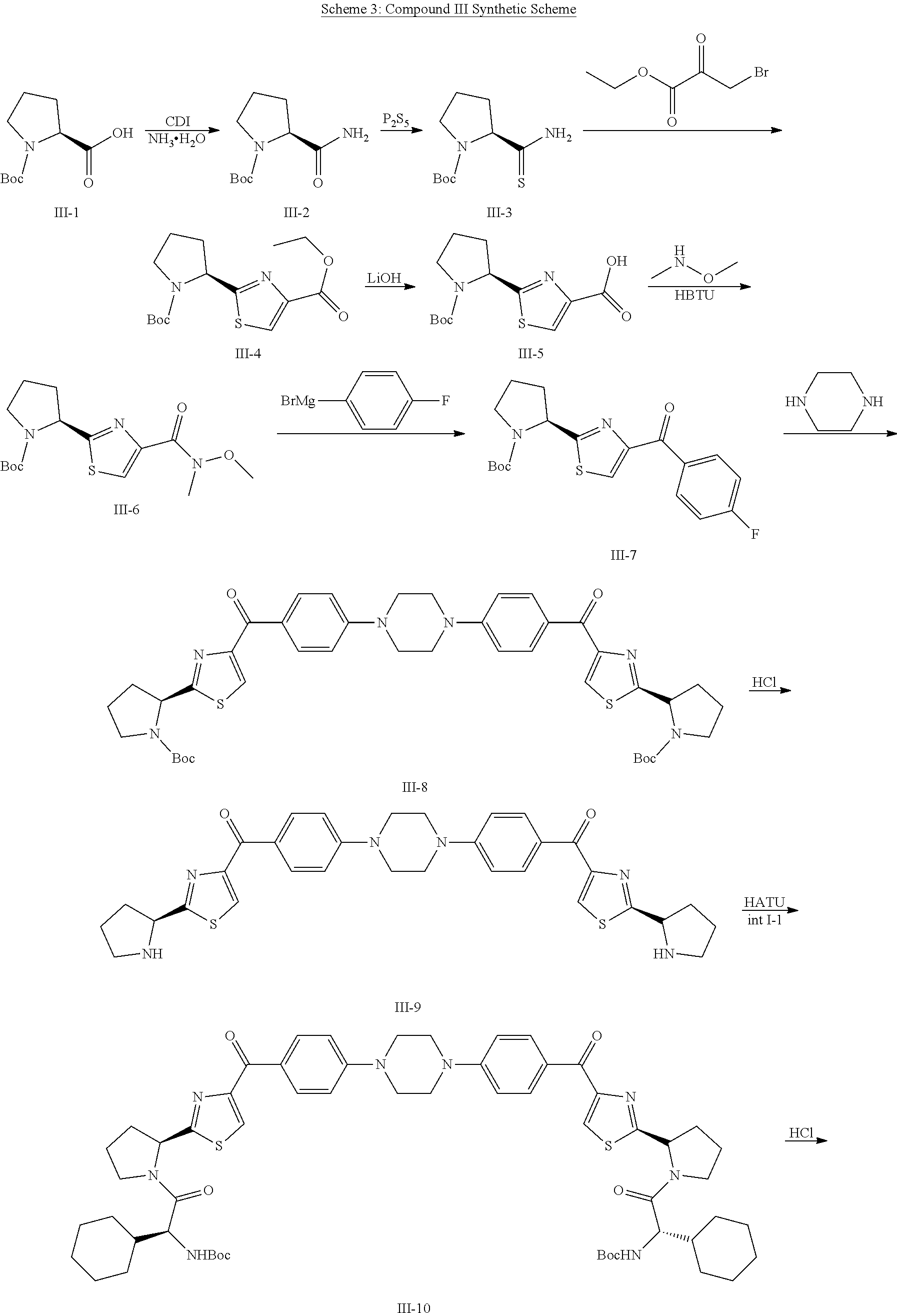

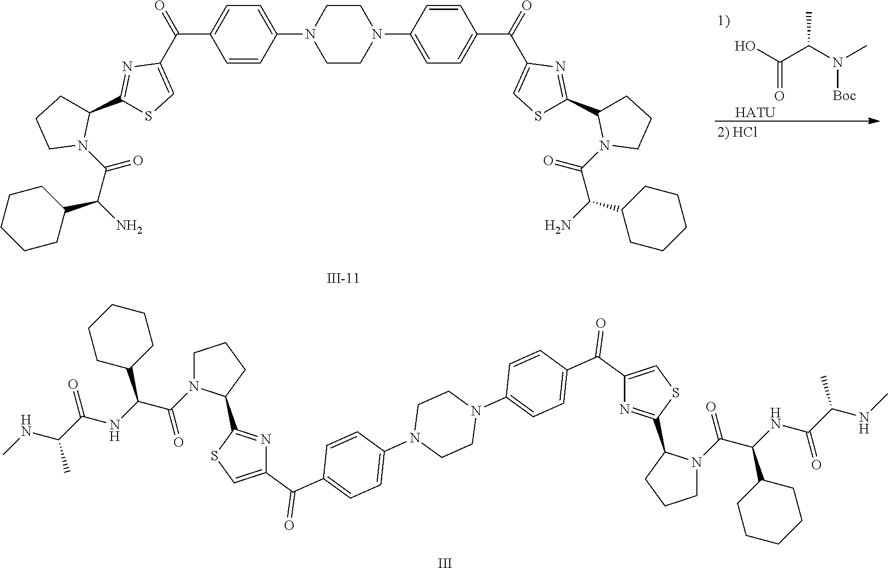

### Example 3: Synthesis of Compound III (Scheme 3)

Compound III may be prepared according to Scheme 3. The Boc-Pro amide (HI-2) is formed from the reaction of Boc-Pro with ammonia and a coupling agent (e.g., carbonyl diimidazole) in water or other suitable solvent. Thiazole ester III-4 may be formed by reacting amide III-3 with P₂S₈ to form the intermediate thioamide II-3, followed by reaction with ethyl 2-oxo-3-bromo-propionate. Hydrolysis of the ethyl ester with (e.g., LiOH) and coupling with N, O-dimethyl hydroxylamine to form the hydroxamate (with e.g., HBTU or other suitable amine coupling reagents) provides the N-Boc hydroxamate III-6. Subsequent reaction with a 4-fluorophenyl Grignard reagent in a suitable solvent (e.g., THF) leads to flurophenylketone III-7. Exposure of compound III-7 to piperazine results in formation the bivalent precursor, III-8. The latter compound may be N-deprotected with acid (e.g., HCl or TFA) which may then be subjected to sequential peptide synthesis conditions to install, e.g., cyclohexylglycine and alanine amino acid derivatives and provide compound III as shown in Scheme 3.

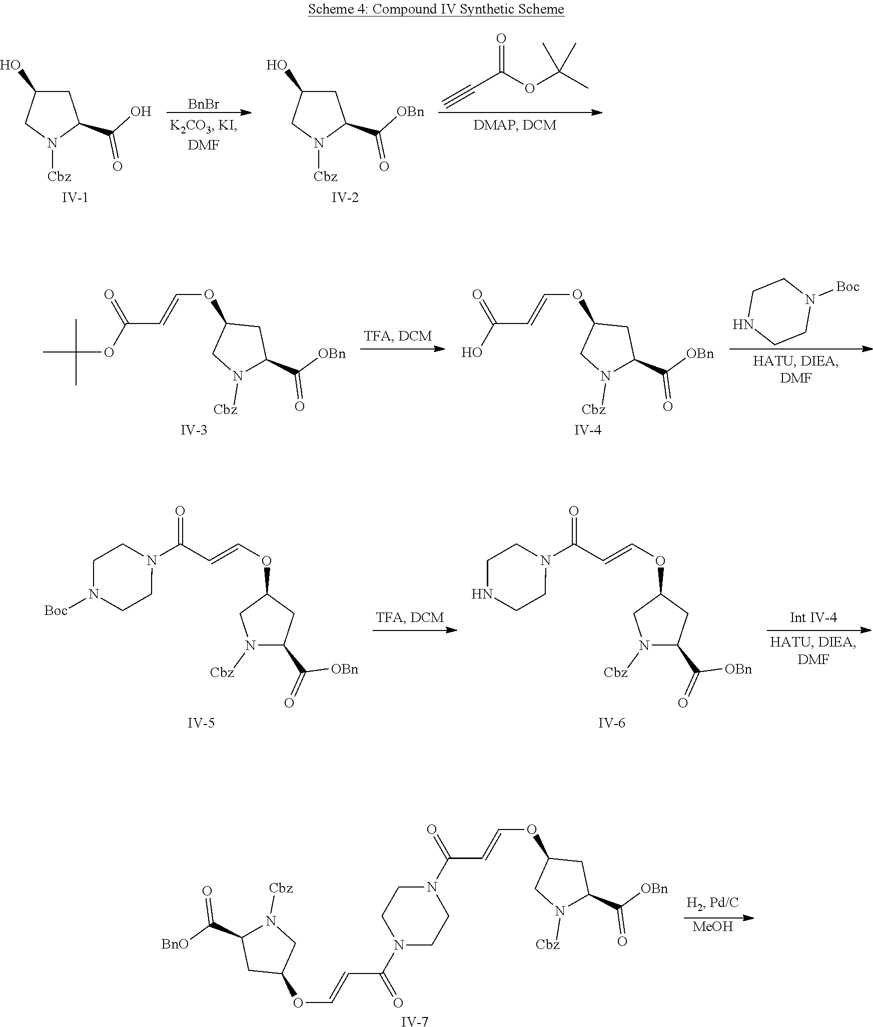

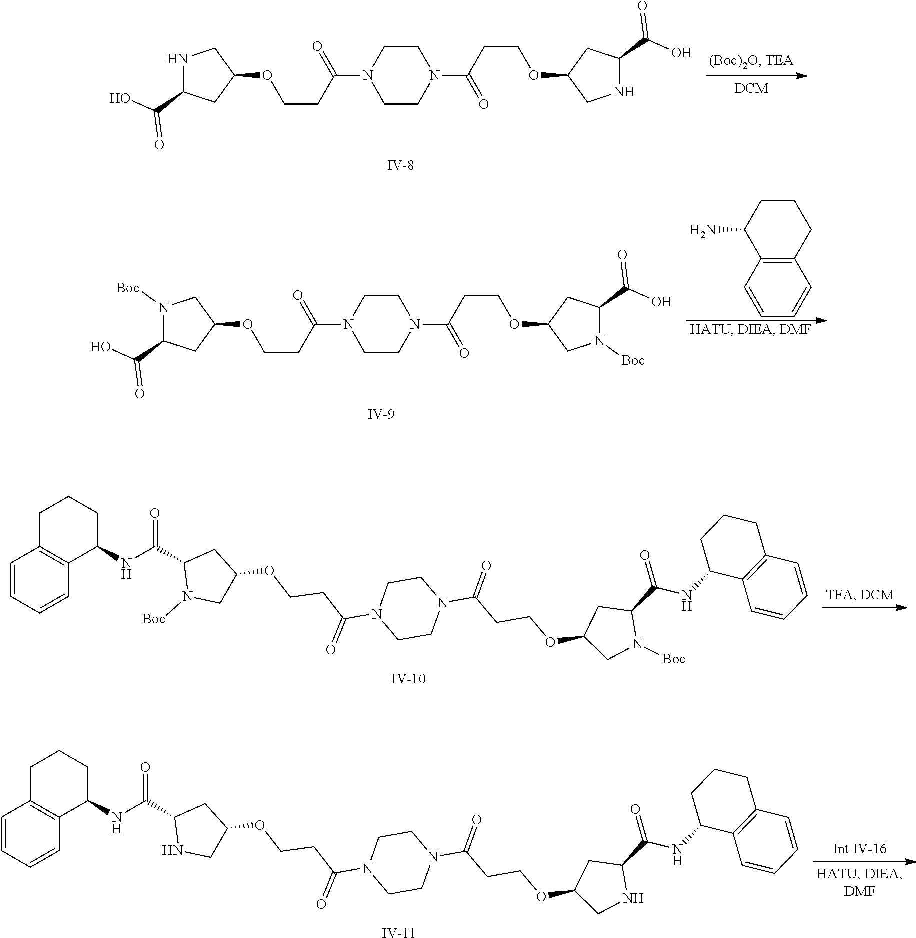

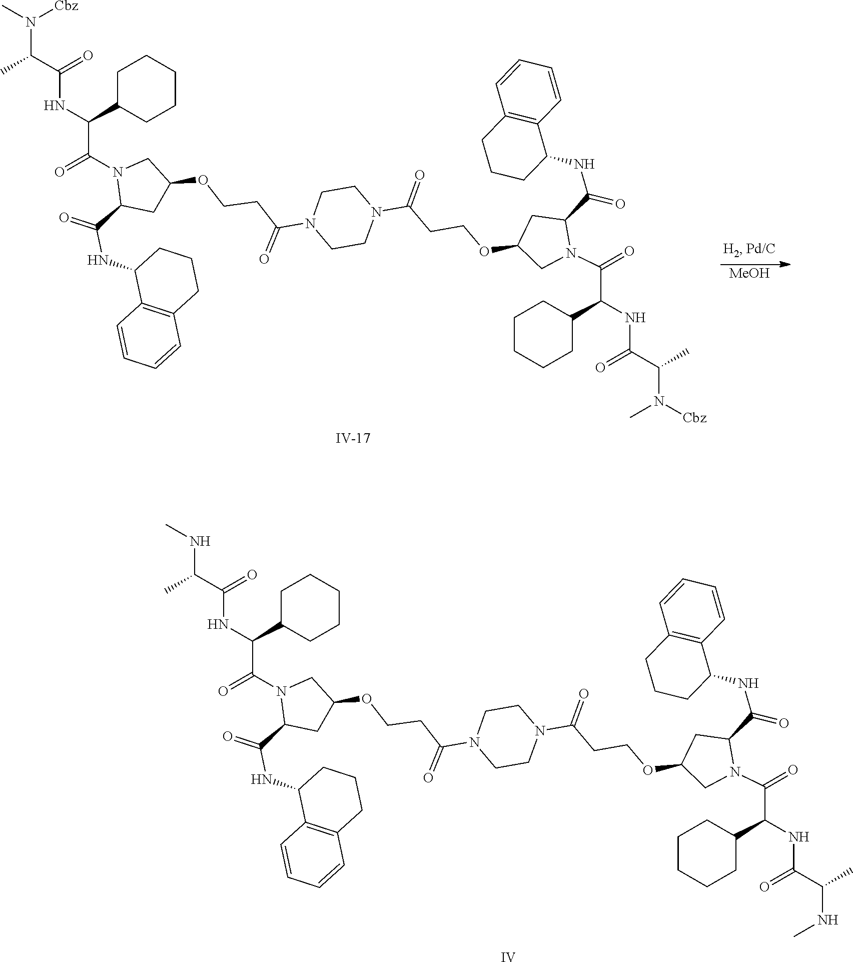 [SMILES: "MeOH" ; C[C@@H](C(=O)N[C@H](C(=O)N1C[C@@H](OCCC(=O)N2CCN(C(=O)CCO[C@H]3C[C@@H](C(=O)N[C@@H]4CCCc5ccccc54)N(C(=O)[C@@H](NC(=O)[C@H](C)N(C)C(=O)OCc4ccccc4)C4CCCCC4)C3)CC2)C[C@H]1C(=O)N[C@@H]1CCCc2ccccc21)C1CCCCC1)N(C)C(=O)OCc1ccccc1 ; "IV-17" ; CN[C@@H](C)C(=O)N[C@H](C(=O)N1C[C@@H](OCCC(=O)N2CCN(C(=O)CCO[C@H]3C[C@@H](C(=O)N[C@@H]4CCCc5ccccc54)N(C(=O)[C@@H](NC(=O)[C@H](C)NC)C4CCCCC4)C3)CC2)C[C@H]1C(=O)N[C@@H]1CCCc2ccccc21)C1CCCCC1 ; "IV"]

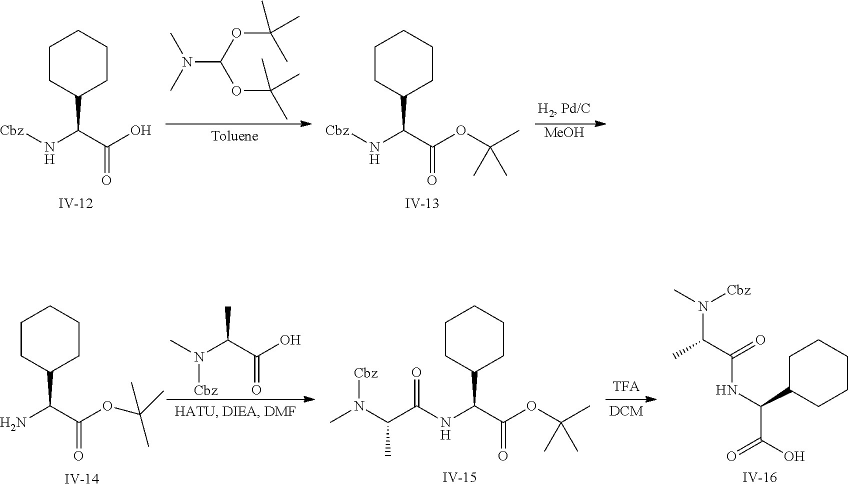

### Example 4: Synthesis of Compound IV (Scheme 4)

(2S, 4S)-Dibenzyl 4-hydroxypyrrolidine-1, 2-dicarboxylate (Compound IV-2): To a solution of (2S, 4S)-1-(benzyloxycarbonyl)-4-hydroxypyrrolidine-2-carboxylic acid (9.5 g, 35.74 mmol) in DMF (100 mL) was added K₂CO₃ (10.8 g, 78.63 mmol) and KI (0.6 g, 3.57 mmol). Then BnBr (18.2 g, 107.21 mmol) was added dropwise to the mixture at 0° C. The resulting mixture was stirred at room temperature for 16 h. The reaction mixture was diluted with EtOAc. The resulted mixture was washed with brine, dried over anhydrous sodium sulfate and filtered. The filtrate was concentrated under vacuum. The residue was purified by flash column chromatography with EtOAc/petroleum ether (1:1, v/v) to afford the title compound (10.0 g, 78%) as a yellow oil. LCMS (ESI, m/z): [M+H]⁺=356.3.

(2S,4S,E)-Dibenzyl-4-(3-tert-butoxy-3-oxoprop-1-enyloxy)pyrrolidine-1,2-dicarboxylate (Compound IV-3): To a solution of Compound IV-2 (10.0 g, 28.17 mmol) in DCM (150 mL) was added DMAP (6.8 g, 56.34 mmol). Then tert-butyl prop-2-ynoate (4.3 g, 33.80 mmol) was added dropwise to the mixture at 0° C. The resulting mixture was stirred at room temperature for 2.5 h. The reaction mixture was diluted with DCM. The resulted mixture was washed with brine, dried over anhydrous sodium sulfate and filtered. The filtrate was concentrated under vacuum. The residue was purified by flash column chromatography with EtOAc/petroleum ether (1:1, v/v) to afford the title compound (9.0 g, 66%) as a yellow oil. LCMS (ESI, m/z): [M+H]⁺=482.2.

(E)-3-((3S, 5S)-1,5-bis(Benzyloxycarbonyl)pyrrolidin-3-yloxy)acrylic acid (Compound IV-4): To a solution of Compound IV-3 (4.8 g, 10.08 mmol) in DCM (30 mL) was added TFA (4 mL). The resulting mixture was stirred at room temperature for 1 h. The reaction mixture was diluted with H₂O and extracted with DCM. The combined organic layer was washed with brine, dried over Na₂SO₄ and filtered. The filtrate was evaporated in vacuo to afford the title compound (4.3 g, crude) as a yellow oil. LCMS (ESI, m/z): [M+H]⁺=426.1.

(2S,4S,E)-Dibenzyl-4-(3-(4-(tert-butoxycarbonyl)piperazin-1-yl)-3-oxoprop-1-enyloxy)pyrrolidine-1,2-dicarboxylate (Compound IV-5): To a solution of Compound IV-4 (2.0 g, 4.70 mmol) in DMF (20 mL) was added HATU (2.2 g, 5.64 mmol) and DIEA (1.8 g, 14.10 mmol) at 0° C. After stirring for 30 min at 0° C., tert-butyl piperazine-1-carboxylate (1.0 g, 5.64 mmol) was added to the mixture. The resulting mixture was stirred at room temperature for 1 h. The reaction mixture was diluted with H₂O and extracted with EtOAc. The combined organic layer was washed with brine, dried over dried over anhydrous sodium sulfate and filtered. The filtrate was concentrated under vacuum. The residue was purified by flash column chromatography with DCM/MeOH (10:1, v/v) to afford the title compound (1.8 g, 64%) as a light yellow oil. LCMS (ESI, m/z): [M+H]⁺=594.3.

(2S,4S,E)-Dibenzyl-4-(3-oxo-3-(piperazin-1-yl)prop-1-enyloxy)pyrrolidine-1,2-dicarboxylate (Compound IV-6): To a solution of Compound IV-5 (1.7 g, 2.96 mmol) in DCM (20 mL) was added TFA (5 mL). The resulting mixture was stirred at room temperature for 1 h. The pH value of the mixture was adjusted to 7 with NaOH (2 N). The reaction mixture was diluted with H₂O and extracted with EtOAc. The organic layer was washed with brine, dried over dried over anhydrous sodium sulfate and filtered. The filtrate was concentrated under vacuum to afford the title compound (1.5 g, crude) as a light yellow oil. LCMS (ESI, m/z): [M+H]⁺=494.3.

(2S,2'S,4S,4'S)-Tetrabenzyl-4,4′-(1E,1′E)-3,3′-(piperazine-1,4-diyl)bis(3-oxoprop-1-ene-3,1-diyl)bis(oxy)dipyrrolidine-1,2-dicarboxylate (Compound IV-7): To a solution of Compound IV-4 (1.0 g, 2.47 mmol) in DMF (20 mL) was added HATU (1.1 g, 2.96 mmol) and DIEA (956.9 mg, 7.40 mmol) at 0° C. After stirring for 30 min, Compound IV-6 (1.5 g, 3.04 mmol) was added to the reaction mixture. The resulting mixture was stirred at room temperature for 1 h. The mixture was diluted with H₂O and extracted with EtOAc. The organic layer was washed with brine, dried over dried over anhydrous sodium sulfate and filtered. The filtrate was concentrated under vacuum. The residue was purified by flash column chromatography with DCM/MeOH (10:1, v/v) to afford the title compound (1.0 g, 45%) as a light yellow oil. LCMS (ESI, m/z): [M+H]⁺=901.3.

(2S,4S)-4-{3-[4-(3-{[(3S,5S)-5-Carboxypyrrolidin-3-yl]oxy}propanoyl)piperazin-1-yl]-3-oxopropoxy}pyrrolidine-2-carboxylic acid (Compound IV-8): To a solution of Compound IV-7 (732 mg, 0.83 mmol) in MeOH (10 mL) was added Pd/C (172.9 mg, 1.62 mmol). The resulting mixture was stirred at room temperature for 16 h under H₂ atmosphere. After the reaction was completed, the reaction mixture was filtered. The filtrate was concentrated under vacuum to afford the title compound (300 mg, crude) as a light yellow oil. LCMS (ESI, m/z): [M+H]⁺=457.2.

(2S,4S)-1-[(tert-Butoxy)carbonyl]-4-{3-[4-(3-{[(3S,5S)-1-[(tert-butoxy)carbonyl]-5-carboxypyrrolidin-3-yl]oxy}propanoyl)piperazin-1-yl]-3-oxopropoxy}pyrrolidine-2-carboxylic acid (Compound IV-9): To a solution of Compound IV-8 (508 mg, 1.22 mmol) in DCM (10 mL) was added Et₃N (2.0 mL) and Boc₂O (534.3 mg, 2.45 mmol). The resulting mixture was stirred at room temperature for 16 h. The mixture was concentrated under vacuum. The residue was purified by flash column chromatography with DCM/MeOH (10:1, v/v) to afford the title compound (650 mg, 81%) as a colorless oil. LCMS (ESI, m/z): [M+H]⁺=657.2.

tert-Butyl (2S,4S)-4-{3-[4-(3-{[(3S,5S)-1-[(tert-butoxy)carbonyl]-5-{[(1R)-1,2,3,4-tetrahydronaphthalen-1-yl]carbamoyl}pyrrolidin-3-yl]oxy}propanoyl)piperazin-1-yl]-3-oxopropoxy}-2-{[(1R)-1,2,3,4-tetrahydronaphthalen-1-yl]carbamoyl}pyrrolidine-1-carboxylate (Compound IV-10): To a solution of Compound IV-9 (650 mg, 0.99 mmol) in DMF (20 mL) was added HATU (903.2 mg, 2.38 mmol) and DIEA (767.5 mg, 5.94 mmol) at 0° C. After stirring for 30 min, (R)-1,2,3,4-tetrahydronaphthalen-1-amine (349.7 mg, 2.38 mmol) was added to the mixture. The resulting mixture was stirred at room temperature for 1 h. The reaction mixture was diluted with H₂O and extracted with EtOAc. The organic layer was washed with brine, dried over anhydrous sodium sulfate and filtered. The filtrate was concentrated under vacuum. The residue was purified by flash column chromatography with MeOH/DCM (1:10, v/v) to afford the title compound (320 mg, 35%) as a colorless oil. LCMS (ESI, m/z): [M+H]⁺=915.6.

(2S,4S)-4-{3-Oxo-3-[4-(3-{[(3S,5S)-5-{[(1R)-1,2,3,4-tetrahydronaphthalen-1-yl]carbamoyl}pyrrolidin-3-yl]oxy}propanoyl)piperazin-1-yl]propoxy}-N-[(1R)-1,2,3,4-tetrahydronaphthalen-1-yl]pyrrolidine-2-carboxamide (Compound IV-11): To a solution of Compound IV-10 (100 mg, 0.11 mmol) in DCM (10 mL) was added TFA (2 mL). The resulting mixture was stirred at room temperature for 1 h. The pH value of the mixture was adjusted to 7 with NaOH (2 N). The reaction mixture was diluted with H₂O and extracted with EtOAc. The organic layer was washed with brine, dried over dried over anhydrous sodium sulfate and filtered. The filtrate was concentrated under vacuum to afford the title compound (72 mg, 92%) as a light yellow oil. LCMS (ESI, m/z): [M+H]⁺=715.3.

Benzyl N-[(1S)-1-{[(1S)-2-[(2S,4S)-4-{3-[4-(3-{[(3S,5S)-1-[(2S)-2-[(2S)-2-{[(benzyloxy)carbonyl](methyl)amino}propanamido]-2-cyclohexylacetyl]-5-{[(1R)-1,2,3,4-tetrahydronaphthalen-1-yl]carbamoyl}pyrrolidin-3-yl]oxy}propanoyl)piperazin-1-yl]-3-oxopropoxy}-2-{[(1R)-1,2,3,4-tetrahydronaphthalen-1-yl]carbamoyl}pyrrolidin-1-yl]-1-cyclohexyl-2-oxoethyl]carbamoyl}ethyl]-N-methylcarbamate (Compound IV-17): To a solution of Compound IV-16 (289.6 mg, 0.77 mmol) in DMF (20 mL) was added HATU (351 mg, 0.93 mmol) and DIEA (129.2 mg, 2.31 mmol) at 0° C. After stirring for 30 min, Compound IV-11 (275 mg, 0.36 mmol) was added to the reaction mixture. The resulting mixture was stirred at room temperature for 1 h. The reaction mixture was diluted with H₂O and extracted with EtOAc. The organic layer was washed with brine, dried over anhydrous sodium sulfate and filtered. The filtrate was concentrated under vacuum. The residue was purified by flash column chromatography with MeOH/DCM (1:10, v/v) to afford the title compound (70 mg, 5%) as a colorless oil. LCMS (ESI, m/z): [M+H]⁺=1431.5.

(2S,4S)-1-[(2S)-2-Cyclohexyl-2-[(2S)-2-(methylamino)propanamido]acetyl]-4-{3-[4-(3-{[(3S,5S)-1-[(2S)-2-cyclohexyl-2-[(2S)-2-(methylamino)propanamido]acetyl]-5-{[(1R)-1,2,3,4-tetrahydronaphthalen-1-yl]carbamoyl}pyrrolidin-3-yl]oxy}propanoyl)piperazin-1-yl]-3-oxopropoxy}-N-[(1R)-1,2,3,4-tetrahydronaphthalen-1-yl]pyrrolidine-2-carboxamide (Compound IV): To a solution of Compound IV-17 (70 mg, 0.05 mmol) in MeOH (10 mL) was added Pd/C (30 mg, 0.28 mmol). The resulting mixture was stirred at room temperature for 16 h under H₂ atmosphere. After the reaction was completed, the mixture was filtered. The filtrate was concentrated under vacuum. The residue was purified by Prep-HPLC with the following conditions: 1) Column: XSelect CSH C18 Column 19×150, 5 um; Mobile Phase A: Water (0.1% FA), Mobile Phase B: ACN; Flow rate: 25 mL/min; Gradient: 11% B to 33% B in 7 min; 254/220 nm; Rt: 6.8 min 2) Column: XSelect CSH C18 Column 19×150.5 um; Mobile Phase A: Water (10 mmol/L NH₄HCO₃), Mobile Phase B: ACN; Flow rate: 25 mL/min; Gradient: 30% B to 55% B in 12 min; 254/220 nm; Rt: 10 min to afford the title compound (4.6 mg, 8%) as a white solid. LCMS (ESI, m/z): [M+H]⁺=1163.5. ¹H NMR (400 MHz, DMSO-d₆, ppm): δ 8.16 (s, 2H), 8.15-7.99 (m, 1H), 7.85-7.40 (m, 1H), 7.52-7.48 (m, 1H), 7.25-7.06 (m, 5H), 4.99-4.81 (m, 2H), 4.31-4.01 (m, 5H), 3.12-2.98 (m, 2H), 3.61-3.32 (m, 3H), 2.23-2.08 (m, 6H), 1.83-1.58 (m, 15H), 1.16-0.96 (m, 12H).

(S)-tert-Butyl-2-(benzyloxycarbonylamino)-2-cyclohexylacetate (Compound IV-13): To a solution of (S)-2-(benzyloxycarbonylamino)-2-cyclohexylacetic acid (400 mg, 1.37 mmol) in toluene (10 mL) was added di-tert-butoxy-N, N-dimethylmethanamine (1.2 g, 5.90 mmol). The resulting mixture was stirred at 110° C. for 16 h under N₂ atmosphere. The reaction mixture was concentrated under vacuum. The residue was purified by flash column chromatography with MeOH/DCM (1:10, v/v) to afford the title compound (246 mg, 51.6%) as a colorless oil. LCMS (ESI, m/z): [M+H]⁺=347.3.

(S)-tert-Butyl 2-amino-2-cyclohexylacetate (Compound IV-14): To a solution of Compound IV-13 (246 mg, 0.75 mmol) in MeOH (10 mL) was added Pd/C (120 mg, 1.23 mmol). The resulting mixture was stirred at room temperature for 16 h under H₂ atmosphere. After the reaction was completed, the mixture was filtered. The filtrate was concentrated under vacuum to afford the title compound (170 mg, crude) as a colorless oil. LCMS (ESI, m/z): [M+H]⁺=214.3.

(S)-tert-Butyl-2-((S)-2-((benzyloxycarbonyl)(methyl)amino)propanamido)-2-cyclohexylacetate (Compound IV-15): To a solution of (S)-2-((benzyloxycarbonyl)(methyl)amino)propanoic acid (189.1 mg, 0.80 mmol) in DMF (20 mL) was added HATU (363.6 mg, 0.96 mmol) and DIE A (309 mg, 2.39 mmol) at 0° C. After stirring for 30 min, Compound IV-14 (170 mg, 0.80 mmol) was added to the reaction mixture. The resulting mixture was stirred at room temperature for 1 h. The reaction mixture was diluted with H₂O and extracted with EtOAc. The organic layer was washed with brine, dried over anhydrous sodium sulfate and filtered. The filtrate was concentrated under vacuum. The residue was purified by flash column chromatography with MeOH/DCM (1:10, v/v) to afford the title compound (340 mg, 98%) as a light yellow oil. LCMS (ESI, m/z): [M+H]⁺=433.3.

(S)-2-((S)-2-((Benzyloxycarbonyl)(methyl)amino)propanamido)-2-cyclohexylacetic acid (Compound IV-16): To a solution of Compound IV-15 (340 mg, 0.79 mmol) in DCM (20 mL) was added TFA (5 mL). The reaction mixture was stirred at room temperature for 2 h. The reaction mixture was diluted with H₂O and extracted with EtOAc. The organic layer was washed with brine, dried over anhydrous sodium sulfate and filtered. The filtrate was concentrated under vacuum to afford the title compound (290 mg, crude) as a light yellow oil. LCMS (ESI, m/z): [M+H]⁺=377.2.

Following the procedure described above for Scheme 4 and substituting the appropriate reagents, starting materials and purification methods known to those skilled in the art, the following compounds were prepared.

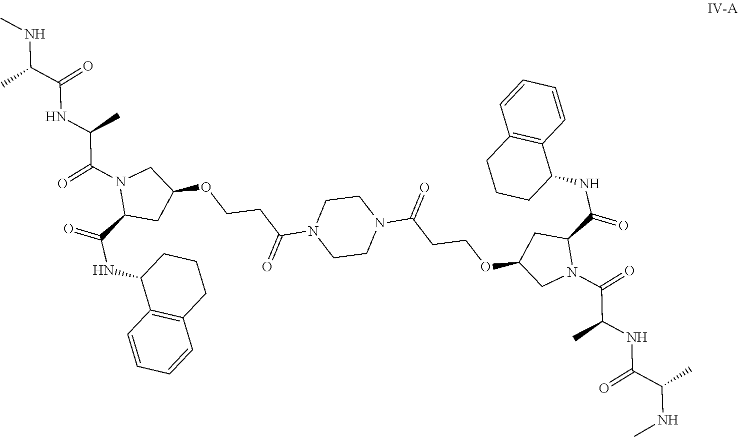 [SMILES: CN[C@@H](C)C(=O)N[C@@H](C)C(=O)N1C[C@@H](OCCC(=O)N2CCN(C(=O)CCO[C@H]3C[C@@H](C(=O)N[C@@H]4CCCc5ccccc54)N(C(=O)[C@H](C)NC(=O)[C@H](C)NC)C3)CC2)C[C@H]1C(=O)N[C@@H]1CCCc2ccccc21]

(2S,4S)-1-[(2S)-2-[(2S)-2-(Methylamino)propanamido]propanoyl]-4-[3-[4-(3-[[(3S,5S)-1-[(2S)-2-[(2S)-2-(methylamino)propanamido]propanoyl]-5-[[(1R)-1,2,3,4-tetrahydronaphthalen-1-yl]carbamoyl]pyrrolidin-3-yl]oxy]propanoyl)piperazin-1-yl]-3-oxopropoxy]-N-[(1R)-1,2,3,4-tetrahydronaphthalen-1-yl]pyrrolidine-2-carboxamide (Compound IV-A):

LCMS (ESI, m/z): [M+H]⁺=1027.6. ¹H NMR (400 MHz, CD₃OD-d₄, ppm): δ 7.39-7.29 (m, 1H), 7.29-7.20 (m, 1H), 7.19-7.01 (m, 6H), 5.15-4.95 (m, 2H), 4.68-4.34 (m, 4H), 4.28-4.09 (m, 2H), 3.99-3.36 (m, 13H), 3.20-2.95 (m, 3H), 2.87-2.65 (m, 4H), 2.60-2.36 (m, 5H), 2.36-2.22 (m, 6H), 2.22-2.09 (m, 3H), 2.08-1.63 (m, 8H), 1.47-1.12 (m, 10H), 1.10-1.00 (m, 2H).

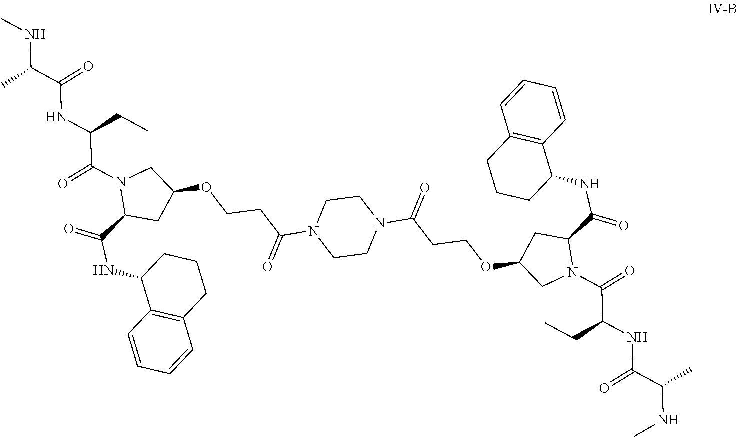 [SMILES: CC[C@H](NC(=O)[C@H](C)NC)C(=O)N1C[C@@H](OCCC(=O)N2CCN(C(=O)CCO[C@H]3C[C@@H](C(=O)N[C@@H]4CCCc5ccccc54)N(C(=O)[C@H](CC)NC(=O)[C@H](C)NC)C3)CC2)C[C@H]1C(=O)N[C@@H]1CCCc2ccccc21]

(2S, 4S)-1-[(2S)-2-[(2S)-2-(Methylamino)propanamido]butanoyl]-4-[3-[4-(3-[[(3S,5S)-1-[(2S)-2-[(2S)-2-(methylamino)propanamido]butanoyl]-5-[[(1R)-1,2,3,4-tetrahydronaphthalen-1-yl]carbamoyl]pyrrolidin-3-yl]oxy]propanoyl)piperazin-1-yl]-3-oxopropoxy]-N-[(1R)-1,2,3,4-tetrahydronaphthalen-1-yl]pyrrolidine-2-carboxamide (Compound IV-B):

LCMS (ESI, m/z): [M+H]⁺=1055.7. ¹H NMR (400 MHz, CD₃OD-d₄, ppm): δ 7.39-7.16 (m, 2H), 7.16-7.07 (m, 6H), 5.15-5.03 (m, 2H), 4.58-4.50 (m, 3H), 4.37-4.18 (m, 3H), 4.04-3.95 (m, 2H), 3.88-3.36 (m, 11H), 3.21-3.10 (m, 2H), 3.07-2.97 (m, 1H), 2.87-2.71 (m, 4H), 2.58-2.24 (m, 11H), 2.23-2.11 (m, 4H), 2.10-1.62 (m, 13H), 1.28-1.17 (m, 4H), 1.09-0.98 (m, 8H).

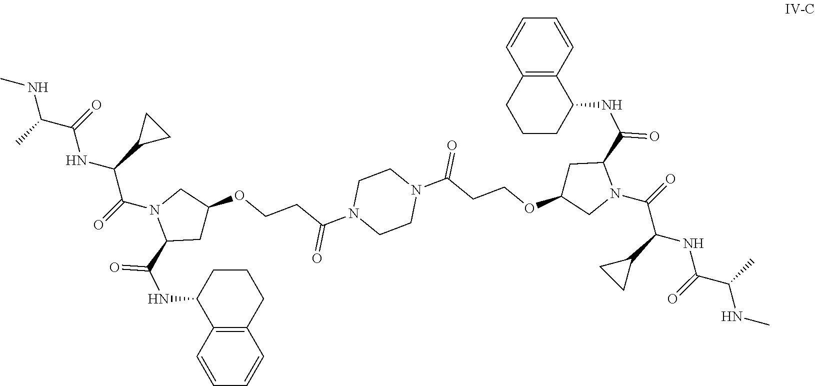

(2S,4S)-1-[(2S)-2-Cyclopropyl-2-[(2S)-2-(methylamino)propanamido]acetyl]-4-{3-[4-(3-{[(3S,5S)-1-[(2S)-2-cyclopropyl-2-[(2S)-2-(methylamino)propanamido]acetyl]-5-{[(1R)-1,2,3,4-tetrahydronaphthalen-1-yl]carbamoyl}pyrrolidin-3-yl]oxy}propanoyl)piperazin-1-yl]-3-oxopropoxy}-N-[(1R)-1,2,3,4-tetrahydronaphthalen-1-yl]pyrrolidine-2-carboxamide (Compound IV-C):

LCMS (ESI, m/z): [M+H]⁺=1081.6. ¹H NMR (400 MHz, CD₃OD-d₄, ppm): δ 7.42-7.21 (m, 2H), 7.16-7.07 (m, 6H), 5.15-5.03 (m, 3H), 4.87-4.56 (m, 2H), 4.32-4.12 (m, 4H), 4.02-3.89 (m, 2H), 3.87-3.37 (m, 11H), 3.19-3.07 (m, 2H), 2.84-2.72 (m, 4H), 2.58-2.31 (m, 6H), 2.30-2.18 (m, 8H), 2.06-1.72 (m, 8H), 1.22-1.18 (m, 8H), 0.54-0.42 (m, 8H).

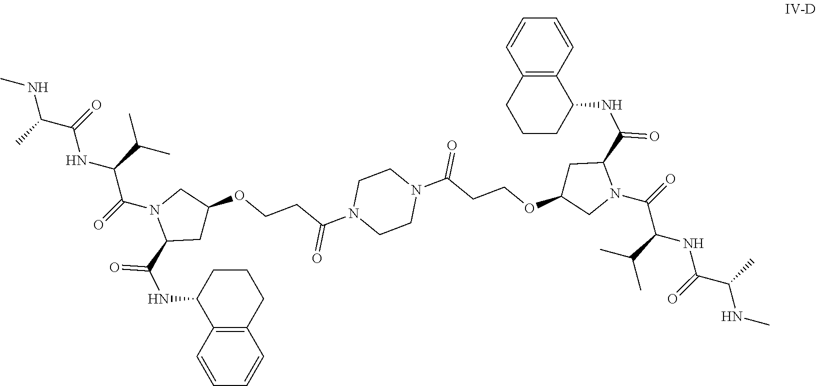

(2S, 4S)-1-[(2S)-3-Methyl-2-[(2S)-2-(methylamino)propanamido]butanoyl]-4-[3-[4-(3-[[(3S,5S)-1-[(2S)-3-methyl-2-[(2S)-2-(methylamino)propanamido]butanoyl]-5-[[(1R)-1,2,3,4-tetrahydronaphthalen-1-yl]carbamoyl]pyrrolidin-3-yl] oxy] propanoyl)piperazin-1-yl]-3-oxopropoxy]-N-[(1R)-1,2,3,4-tetrahydronaphthalen-1-yl]pyrrolidine-2-carboxamide (Compound IV-D):

LCMS (ESI, m/z): [M+H]⁺=1083.5. ¹H NMR (400 MHz, CD₃OD-d₄, ppm): δ 7.45-7.25 (m, 2H), 7.15-7.10 (m, 4H), 7.09-7.07 (m, 2H), 5.06-5.03 (m, 2H), 4.70-4.52 (m, 2H), 4.50-4.43 (m, 4H), 4.29-4.16 (m, 2H), 4.12-4.03 (m, 2H), 3.79-3.65 (m, 7H), 3.64-3.37 (m, 5H), 3.22-3.12 (m, 2H), 2.87-2.71 (m, 4H), 2.57-2.38 (m, 6H), 2.32 (s, 6H), 2.27-2.15 (m, 4H), 2.07-1.70 (m, 8H), 1.24 (d, J=7.2 Hz, 6H), 1.04 (d, J=6.4 Hz, 6H), 0.99-0.96 (m, 6H).

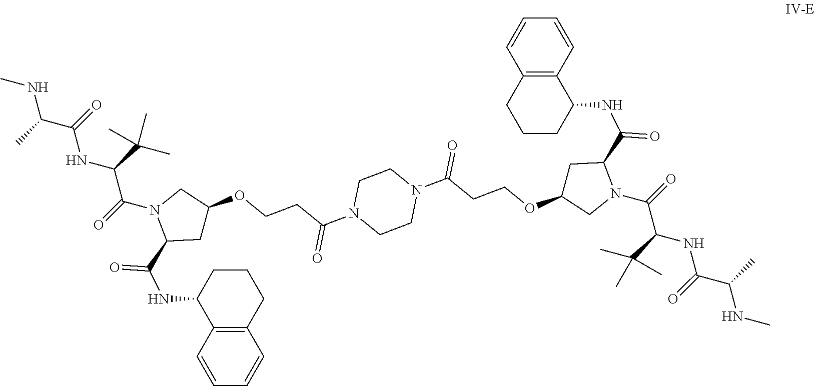

(S,S,2S,2'S,4S,4'S)-4,4′-((Piperazine-1,4-diylbis(3-oxopropane-3,1-diyl))bis(oxy))bis(1-((S)-3,3-dimethyl-2-((S)-2-(methylamino)propanamido)butanoyl)-N—((R)-1,2,3,4-tetrahydronaphthalen-1-yl)pyrrolidine-2-carboxamide) (Compound IV-E): LCMS (ESI, m/z): [M+H]⁺=1111.7.

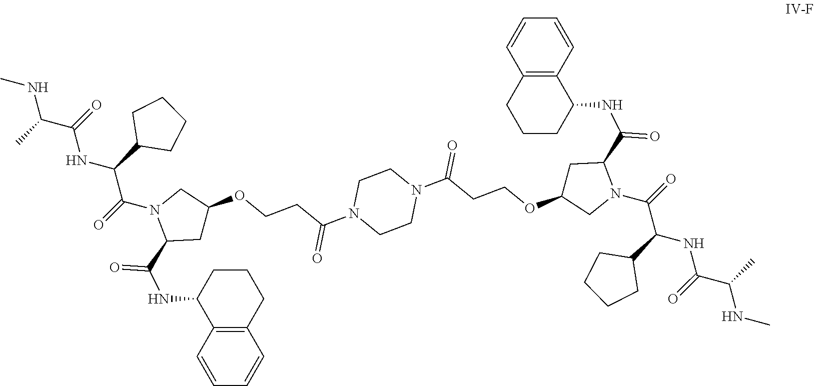

(S,S,2S,2'S,4S,4'S)-4,4′-((Piperazine-1,4-diylbis(3-oxopropane-3,1-diyl))bis(oxy))bis(l-((S)-2-cyclopentyl-2-((S)-2-(methylamino)propanamido)acetyl)-N—((R)-1,2,3,4-tetrahydronaphthalen-1-yl)pyrrolidine-2-carboxamide) (Compound IV-F): LCMS (ESI, m/z): [M+H]⁺=1137.7.

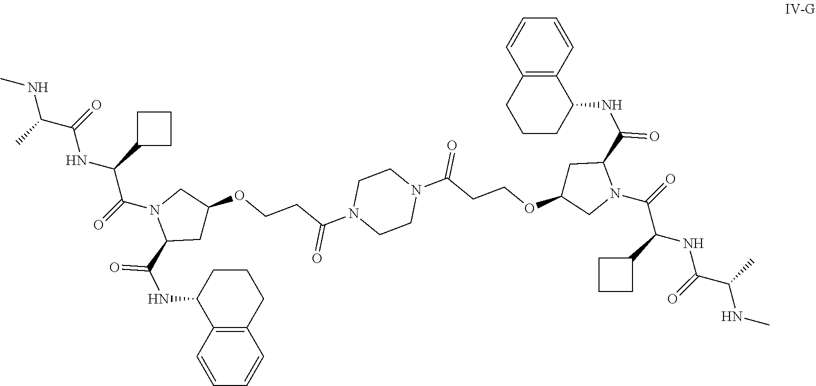

(2S,4S)-1-[(2S)-2-Cyclobutyl-2-[(2S)-2-(methylamino)propanamido]acetyl]-4-[3-[4-(3-[[(3S,5S)-1-[(2S)-2-cyclobutyl-2-[(2S)-2-(methylamino)propanamido]acetyl]-5-[[(1R)-1,2,3,4-tetrahydronaphthalen-1-yl]carbamoyl]pyrrolidin-3-yl] oxy] propanoyl)piperazin-1-yl]-3-oxopropoxy]-N-[(1R)-1,2,3,4-tetrahydronaphthalen-1-yl]pyrrolidine-2-carboxamide (Compound IV-G):

LCMS (ESI, m/z): [M/2+H]⁺=555.4. ¹H NMR (300 MHz, CD₃OD-d₄, ppm): δ 7.39-7.22 (m, 2H), 7.21-7.03 (m, 6H), 5.10-5.00 (m, 2H), 4.77-4.64 (m, 2H), 4.59-4.42 (m, 2H), 4.25-4.16 (m, 2H), 4.03-3.82 (m, 4H), 3.80-3.68 (m, 5H), 3.63-3.52 (m, 2H), 3.51-3.33 (m, 3H), 3.27-3.09 (m, 2H), 2.89-2.70 (m, 6H), 2.59-2.19 (m, 14H), 2.01-1.66 (m, 22H), 1.34-1.17 (m, 6H).

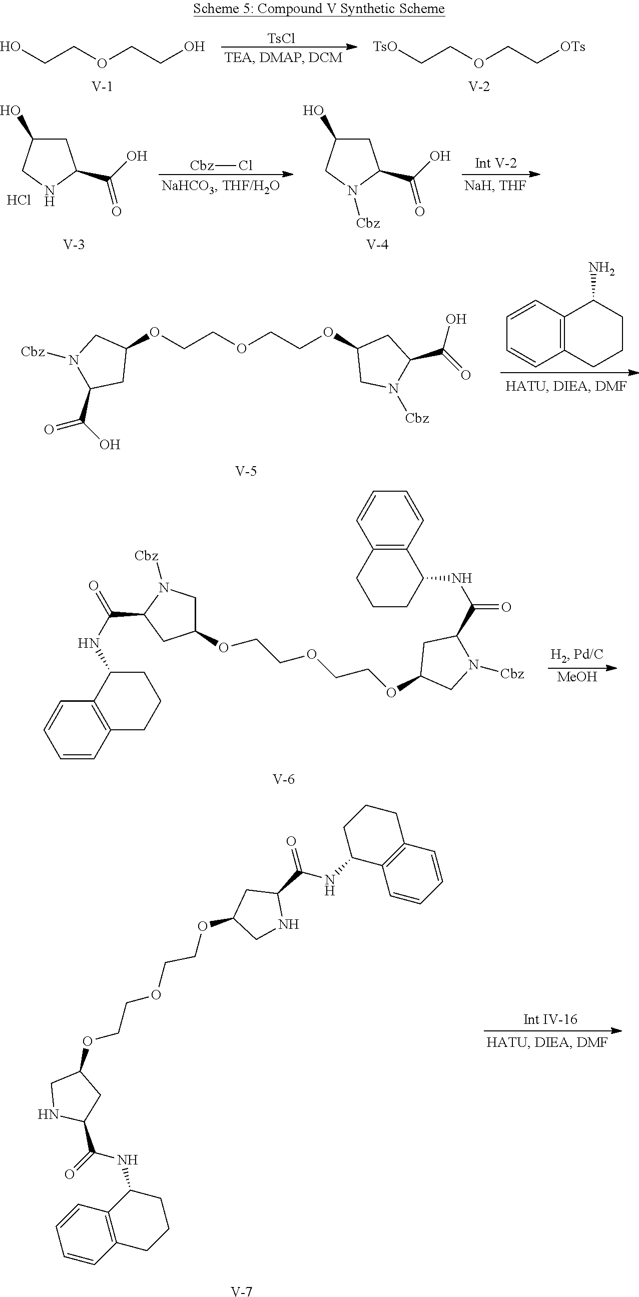

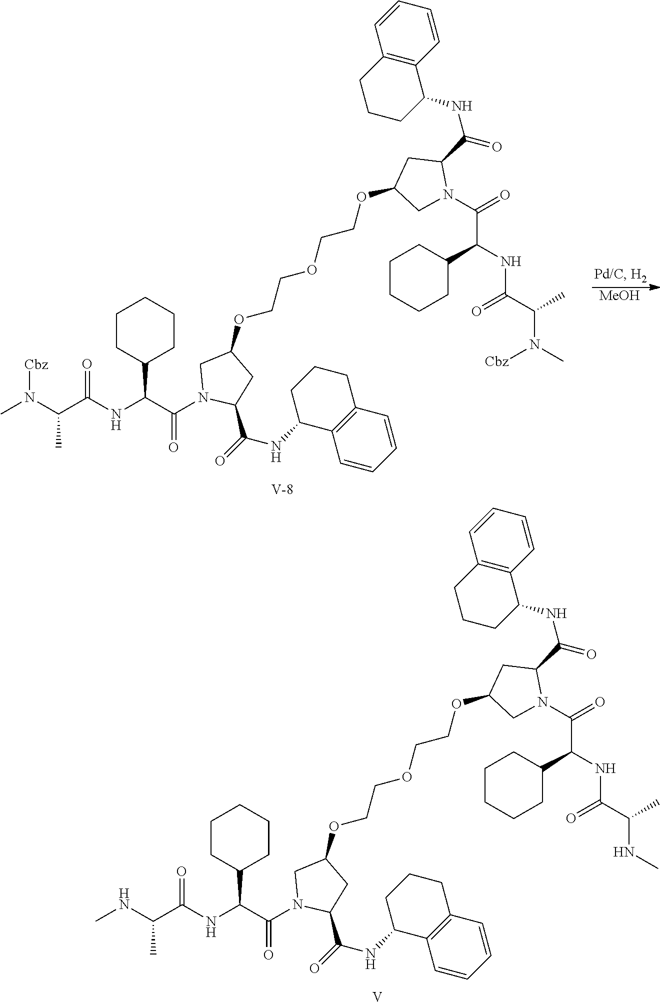

### Example 5: Synthesis of Compound V (Scheme 5)

2-[2-[(4-Methylbenzenesulfonyl)oxy]ethoxy]ethyl 4-methylbenzene-1-sulfonate (Compound V-2): To a solution of 2-(2-hydroxyethoxy)ethan-1-ol (10.5 g, 98.94 mmol) in DCM (300.0 mL) was added DMAP (4.7 g, 38.47 mmol) and TEA (20.3 g, 200.61 mmol). Then 4-methylbenzene-1-sulfonyl chloride (40.3 g, 211.393 mmol) was added to the mixture at 0° C. The mixture was stirred at room temperature for 16 h. The mixture was evaporated in vacuo. The residue was purified by flash column chromatography with DCM/MeOH (99:1, v/v) to afford the title compound (32.1 g, 78%) as a white solid. LCMS (ESI, m/z): [M+H]⁺=415.1

(2S,4S)-1-[(Benzyloxy)carbonyl]-4-hydroxypyrrolidine-2-carboxylic acid (Compound V-4): To a solution of (2S,4S)-4-hydroxypyrrolidine-2-carboxylic acid hydrochloride (10.1 g, 60.38 mmol) in H₂O (100.0 mL) was added NaHCO₃ (18.1 g, 215.46 mmol). Then a solution of benzyl carbonochloridate (12.6 g, 73.86 mmol) in THF (100.0 mL) was added dropwise to the mixture at 0° C. under N₂. The mixture was stirred at room temperature for 16 h. After the reaction was completed, the reaction mixture was extracted with Et₂O. The pH value of the aqueous phase was adjusted to 3-4 with HCl (1M). The resulting mixture was extracted with EtOAc. The combined organic layer was washed with brine, dried over Na₂SO₄ and filtered. The filtrate was evaporated in vacuo to afford the title compound (7.2 g, crude) as a white solid. LCMS (ESI, m/z): [M+H]⁺=266.1

(2S,4S)-1-[(Benzyloxy)carbonyl]-4-[2-(2-[[(3S,5S)-1-[(benzyloxy)carbonyl]-5-carboxypyrrolidin-3-yl]oxy]ethoxy)ethoxy]pyrrolidine-2-carboxylic acid (Compound V-5): To a solution of Compound V-4 (1.9 g, 7.16 mmol) in THF (70.0 mL) was added NaH (832 mg, 20.80 mmol, 60%) at 0° C. under N₂. The mixture was stirred at 0° C. for 30 min. Then a solution of Compound V-2 (1.9 g, 4.60 mmol) in THF (20.0 mL) was added dropwise to the mixture at 0° C. The mixture was stirred at room temperature for 2 days. The pH value of the mixture was adjusted to 4 with HCl (1 mol/L) and then evaporated in vacuo. The residue was purified by reverse phase flash column chromatography with CH₃CN/H₂O (60:40, v/v) to afford the title compound (680 mg, 16%) as a white solid. LCMS (ESI, m/z): [M+H]⁺=601.3

Benzyl (2S,4S)-4-[2-(2-[[(3S,5S)-1-[(benzyloxy)carbonyl]-5-[[(1R)-1,2,3,4-tetrahydronaphthalen-1-yl] carbamoyl] pyrrolidin-3-yl] oxy] ethoxy)ethoxy]-2-[[(1R)-1,2,3,4-tetrahydronaphthalen-1-yl]carbamoyl]pyrrolidine-1-carboxylate (Compound V-6): To a mixture of Compound V-5 (680 mg, 1.13 mmol), (1R)-1,2,3,4-tetrahydronaphthalen-1-amine (519 mg, 3.52 mmol) and DIEA (1.5 mL, 8.61 mmol) in DMF (10.0 mL) was added HATU (2.4 g, 6.41 mmol) at 0° C. under N₂. The mixture was stirred at 0° C. for 2 h. After the reaction was completed, the mixture was purified by reverse phase flash column chromatography with CH₃CN/H₂O (80:20, v/v) to afford the title compound (671.6 mg, 69%) as a light yellow oil. LCMS (ESI, m/z): [M+H]⁺=859.4.

(2S,4S)—N-[(1R)-1,2,3,4-Tetrahydronaphthalen-1-yl]-4-[2-(2-[[(3S,5S)-5-[[(1R)-1,2,3,4-tetrahydronaphthalen-1-yl] carbamoyl] pyrrolidin-3-yl]oxy]ethoxy)ethoxy]pyrrolidine-2-carboxamide (Compound V-7): To a solution of Compound V-6 (781 mg, 0.91 mmol) in MeOH (20.0 mL) was added Pd/C (610 mg, 5.73 mmol). The mixture was stirred at room temperature for 16 h under H₂. After the reaction was completed, the reaction mixture was filtered. The filtrate was evaporated in vacuo to afford the title compound (460 mg, crude) as a light yellow oil. LCMS (ESI, m/z): [M+H]⁺=591.4.

Benzyl N-[(1S)-1-[[(1S)-2-[(2S,4S)-4-[2-(2-[[(3S,5S)-1-[(2S)-2-[(2S)-2-[[(benzyloxy)carbonyl](methyl)amino]propanamido]-2-cyclohexylacetyl]-5-[[(1R)-1,2,3,4-tetrahydronaphthalen-1-yl] carbamoyl] pyrrolidin-3-yl] oxy] ethoxy)ethoxy]-2-[[(1R)-1,2,3,4-tetrahydronaphthalen-1-yl]carbamoyl]pyrrolidin-1-yl]-1-cyclohexyl-2-oxoethyl]carbamoyl]ethyl]-N-methylcarbamate (Compound V-8): To a mixture of Compound V-7 (400 mg, 0.67 mmol), Compound IV-16 (493.6 mg, 1.31 mmol) and DIEA (1.3 mL, 7.46 mmol) in DMF (10.0 mL) was added HATU (888.1 mg, 2.34 mmol) at 0° C. under N₂. The mixture was stirred at 0° C. for 2 h. The mixture was diluted with H₂O and extracted with EtOAc. The combined organic layer was washed with brine, dried over Na₂SO₄ and filtered. The filtrate was evaporated in vacuo. The residue was purified by flash column chromatography with DCM/MeOH (94:6, v/v) and then purified by reverse phase flash column chromatography with CH₃CN/H₂O (98:2, v/v) to afford the title compound (630 mg, 71%) as a light yellow oil. LCMS (ESI, m/z): [M+H]⁺=1307.7.

(2S,4S)-1-[(2S)-2-Cyclohexyl-2-[(2S)-2-(methylamino)propanamido]acetyl]-4-[2-(2-[[(3S,5S)-1-[(2S)-2-cyclohexyl-2-[(2S)-2-(methylamino)propanamido]acetyl]-5-[[(1R)-1,2,3,4-tetrahydronaphthalen-1-yl]carbamoyl]pyrrolidin-3-yl] oxy] ethoxy)ethoxy]-N-[(1R)-1,2,3,4-tetrahydronaphthalen-1-yl] pyrrolidine-2-carboxamide (Compound V): To a solution of Compound V-8 (630 mg, 0.48 mmol) in MeOH (20.0 mL) was added Pd/C (781 mg, 7.34 mmol). The mixture was stirred at room temperature for 16 h under H₂. After the reaction was completed, the reaction mixture was filtered. The filtrate was evaporated in vacuo. The residue was purified by Prep-HPLC with the following conditions: Column: YMC-Actus Triart C18 30×250 mm, 5 um; Mobile Phase A: Water (10 mmol/L NH₄HCO₃), Mobile Phase B: ACN; Flow rate: 60 mL/min; Gradient: 53% B to 69% B in 9 min; 254 nm; Rt: 8.55 min to afford the title compound (23.8 mg, 5%) as an off-white solid. LCMS (ESI, m/z): [M+H]⁺=1040.3. ¹H NMR (400 MHz, CD₃OD-d₄, ppm): δ 7.39-7.29 (m, 2H), 7.17-7.06 (m, 6H), 5.12-5.00 (m, 2H), 4.50-4.45 (m, 4H), 4.22-4.12 (m, 4H), 3.75-3.41 (m, 10H), 3.15-3.09 (m, 2H), 2.90-2.70 (m, 4H), 2.37-2.16 (m, 9H), 2.05-1.57 (m, 20H), 1.22-1.04 (m, 17H).

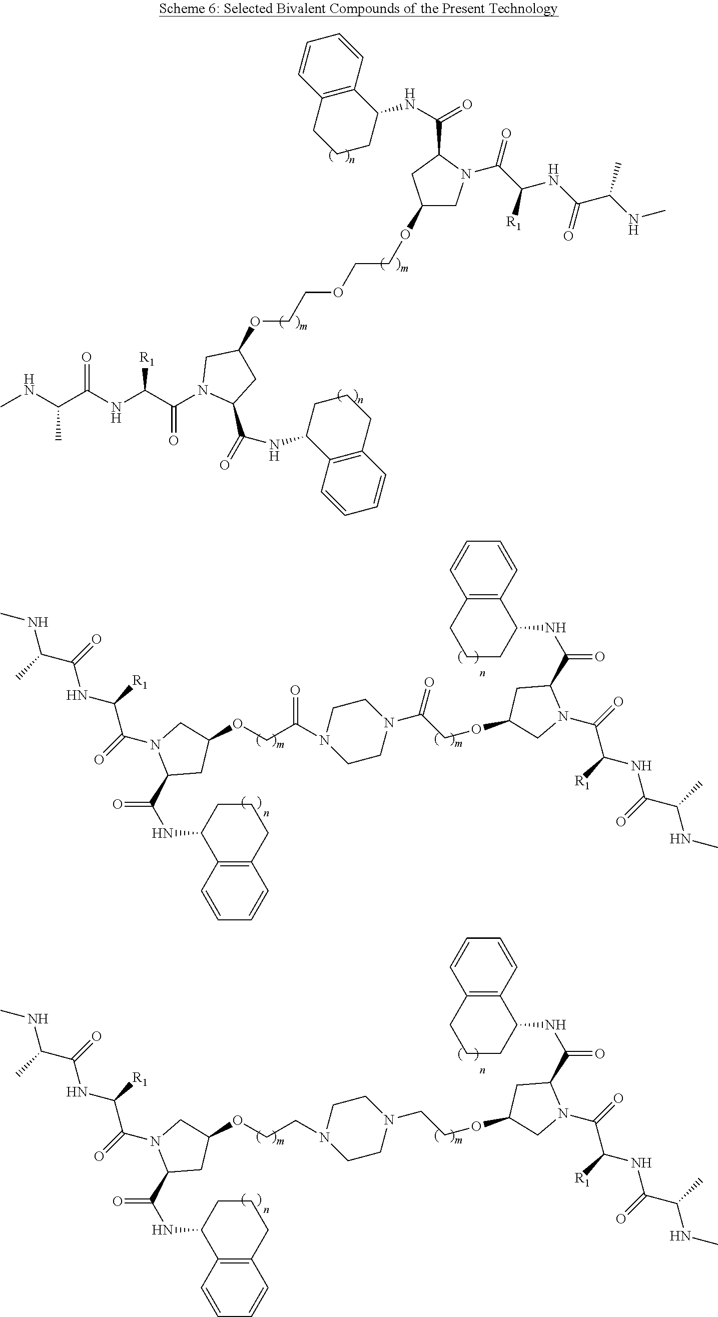

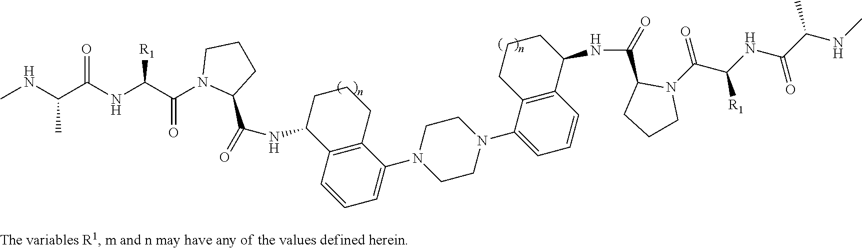

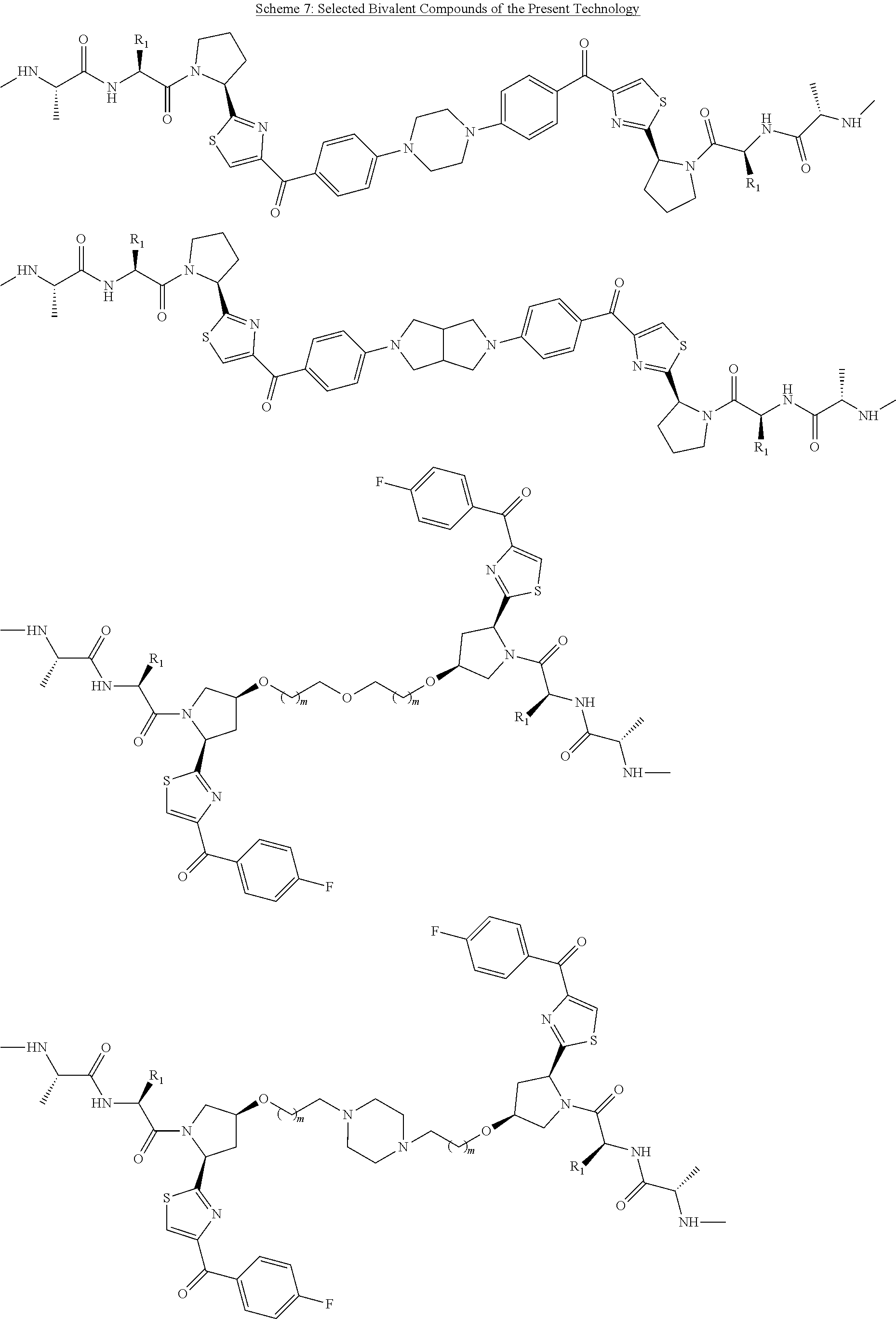

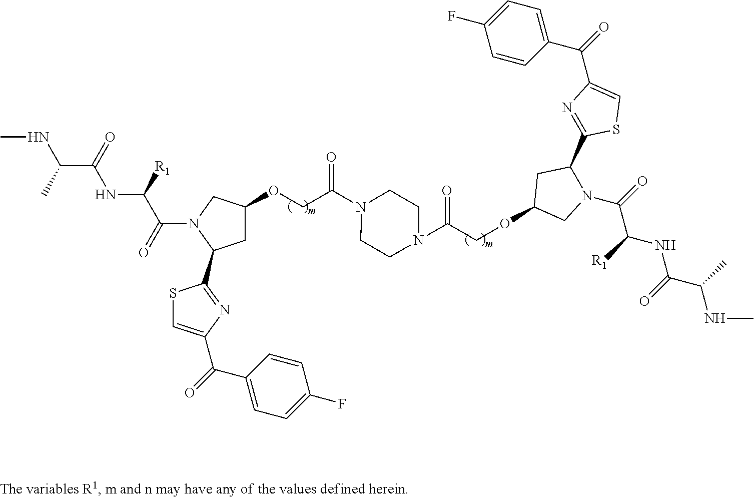

### Example 6: Synthesis of Selected Bivalent Compounds

Following the procedures described above for Scheme 1, Scheme 2, Scheme 4 or Scheme 5 and substituting the appropriate reagents, starting materials and purification methods known to those skilled in the art, the compounds in Scheme 6 may be prepared.

### Example 7: Synthesis of Selected Bivalent Compounds

Following the procedure described above for Scheme 2-5 and substituting the appropriate reagents, starting materials and purification methods known to those skilled in the art, the compounds shown in Scheme 7 may be prepared.

### Example 8: Biological Activity

### Assay Protocol

IAPs are one main cause of cancer development and may result from overexpression of anti-apoptotic proteins. This protocol establishes three binding assays for XIAP Bir3 domain, cIAP1 and cIAP2 using FP (Fluorescence polarization) technology. The fluorescence probe used is a synthetic peptide conjugated to 5-carboxyfluorescein (AbuRPFK-5FAM). The fluorescence polarization value (mP) was detected by Envision, which was used to reflect the binding degree of protein and fluorescent marker. Reagents and equipment used in the assay are listed below, followed by the protocol.

| Number | Name | Vendor | Cat# |
|---|---|---|---|
| 1 | HEPES | Life Technologies | 15630-080 |
| 2 | NaCl | Sigma | S5886 |
| 3 | Triton X-100 | Sigma | T8787 |
| 4 | XIAP-BIR3 | Reaction Biology | APT-11-374 |
| 5 | cIAP1-BIR3 | Reaction Biology | APT-11-370 |
| 6 | cIAP2-BIR3 | Reaction Biology | APT-11-372 |
| 7 | AbuRPF-K(5-Fam)-NH2(SM5F) | NJ Peptide | |
| 8 | DMSO | MP | 196055 |
| 9 | Topseal A | PerkinElmer | E5341 |
| 10 | ProxiPlate-384 F Plus | PerkinElmer | 6008260 |
| 11 | V96 MicroWell Plates | nunc | 249944 |
| 12 | 384-well plates | corning | 3657 |
| 13 | Envision | Perkin Elmer | 2104 |
| 14 | Centrifuge | Eppendorf | 5810R |

a) Prepare 100 times of the final cpd concentration in appropriate tube and transfer 5 uL compound (“cpd”) to 45 μL 1× reaction buffer with 10% DMSO.
b) The final reference cpd concentration is 10000, 3333.3, 1111.1, 370.4, 123.4, 41.2, 13.7, 4.57, 1.52, 0.51, 0.17 and 0 nM. So the 100 times of the concentration is 1000, 333.3, 111.1, 37.04, 12.34, 4.12, 1.0.46, 0.15, 0.05, 0.017 and 0 μM. The final test cpds concentration is 3333.3, 1111.1, 370.4, 123.4, 41.2, 13.7, 4.57, 1.52, 0.51, 0.17, 0.057 and 0 nM. So the 100 times of the concentration is 333.3, 111.1, 37.04, 12.34, 4.12, 1.0.46, 0.15, 0.05, 0.017, 0.0057 and 0 μM
c) Add 8 μL/well each dose enzyme to 384 well microplate (ProxiPlate-384 F Plus, 6008260) using multichannel pipette, prepared in step 2.1.1.2
d) Centrifuge at 1000 rpm.
e) Add 2 μL/well cpd to 384 well microplate (ProxiPlate-384 F Plus, 6008260) using multichannel pipette, prepared in step a).
f) Centrifuge at 1000 rpm. RT, 15 min.
g) Start the assay by adding 10 uL/well substrate (prepared in step 2.1.1.3) to the same 384 well microplate using multichannel pipette
h) Centrifuge at 1000 rpm.
i) Cover the assay plate and incubate for 60 min at 25° C.
j) Read on Envision 2104 for mP and plot the IC₅₀s with mP values.
k) Data analysis: IC50s were determined based on a non-linear regression analysis of data collected.

### Biological Data

Compounds of the present technology as described herein were or are tested according to the protocol above and show or are expected to show IC50 values equal to or below 1 uM in one or more of the above assays. Certain compounds exhibit or are expected to exhibit IC₅₀s of 100 nM or less, and others exhibit or are expected to exhibit IC₅₀s of 10 nM or less in one or more of the above binding assays. Exemplary results are shown in Table 1 for selected compounds.

TABLE 1

| | IC₅₀ (nm) | IC₅₀ (nm) | IC₅₀ (nm) |
|---|---|---|---|
| | XIAP-BIR3 | cIAP1-BIR3 | cIAP2-BIR3 |
| | binding | binding | binding |
| Compound | assay | assay | assay |
| I | B | A | B |
| I-A | C | A | B |
| I-B | C | A | B |
| I-C | B | A | B |
| II | B | A | B |
| II-A | C | A | B |
| II-B | B | A | A |
| II-C | A | A | A |
| II-D | B | A | B |
| II-F | C | A | B |
| II-G | A | A | A |
| II-H | B | A | B |
| IV | B | A | B |
| IV-A | C | A | B |
| IV-B | C | A | B |
| IV-C | C | A | A |
| IV-D | B | A | B |
| IV-G | B | A | B |
| V | B | A | A |

A: 0.1-10 nM
B: >10 nM-100 nM
C: >100 nM-1 uM

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A compound of Formula I, a stereoisomer thereof, or a pharmaceutically acceptable salt of the compound or the stereoisomer of the compound: 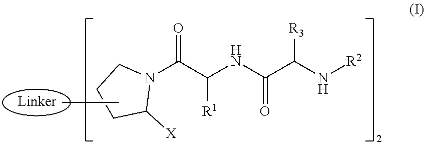
wherein
X is a bond to the Linker or, when the Linker is attached to positions, 2, 3, or 4 on the pyrrolidine ring, X is selected from 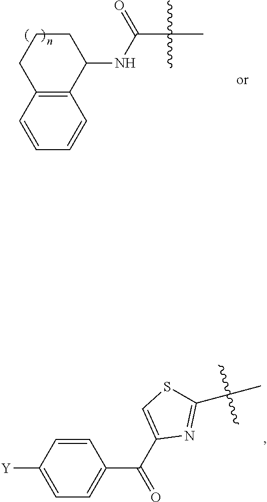
wherein
Y is H or halogen;
R¹ and R³ are independently selected from a substituted or unsubstituted C₁₋₆ alkyl or a C₃₋₆ cycloalkyl group;
R² is H or a substituted or unsubstituted C₁₋₆ alkyl group;
m is 1, 2, 3, 4, 5, or 6;
n is 0, 1 or 2; and
Linker is selected from the group consisting of

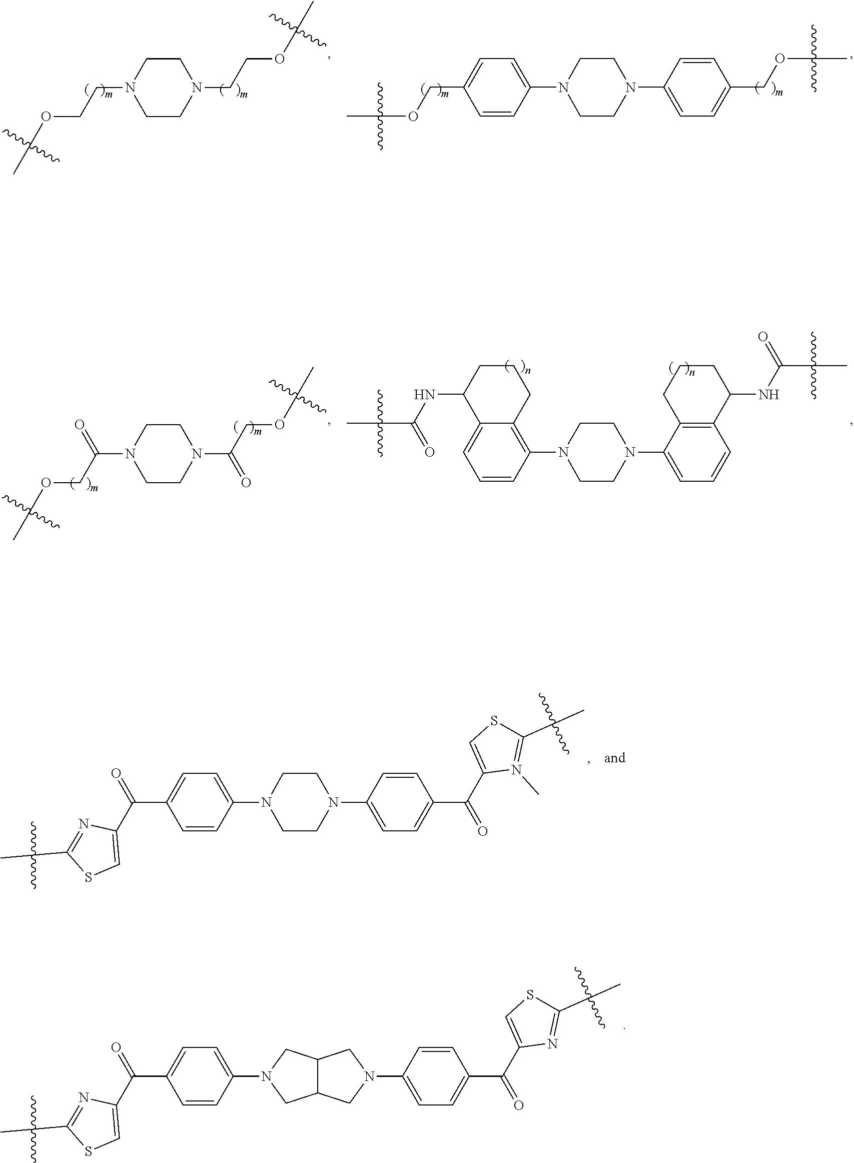

2. The compound of claim 1 wherein Linker is

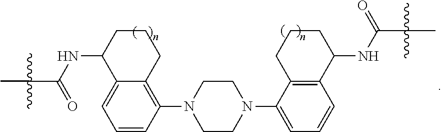

3. The compound of claim 1 wherein n is 1.

4. The compound of claim 1, wherein X is a bond to Linker.

5. The compound of claim 1 wherein Linker is

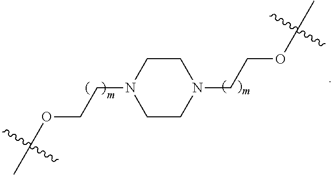

6. The compound of claim 1 wherein m is 1, 2 or 3.

7. The compound of claim 1 wherein Linker is

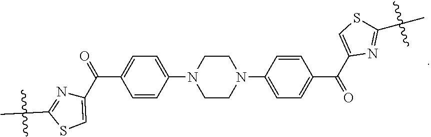 [SMILES: *c1nc(C(=O)c2ccc(N3CCN(c4ccc(C(=O)c5csc(*)n5)cc4)CC3)cc2)cs1]

8. The compound of claim 7 wherein X is a bond to Linker.

9-10. (canceled)

11. The compound of claim 1 wherein Linker is

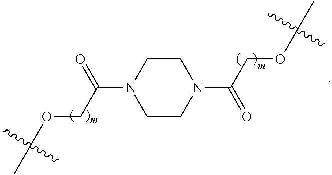

12. The compound of claim 11 wherein m is 1, 2 or 3.

13. The compound of claim 1 wherein Linker is attached to the 3 position of the pyrrolidine of the compound of Formula I.

14. The compound of claim 1 wherein X is 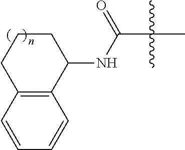
and n is 1.

15. The compound of claim 1 wherein X is 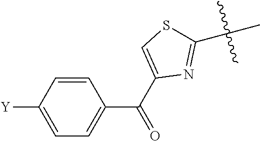
and Y is F.

16. The compound of claim 1 wherein R¹ is a methyl, ethyl, n-propyl, i-propyl, n-butyl, s-butyl, i-butyl, t-butyl, cyclopropyl, cyclobutyl, cyclohexyl, or cyclopentyl group.

17. The compound of claim 1 wherein R² is a methyl, ethyl, n-propyl, i-propyl, n-butyl, i-butyl, or t-butyl group.

18. The compound of claim 1 wherein R³ is a methyl, ethyl, n-propyl, i-propyl, n-butyl, s-butyl, i-butyl, t-butyl, cyclopropyl, cyclobutyl, cyclohexyl, or cyclopentyl group.

19. The compound of claim 1 wherein the compound is selected from

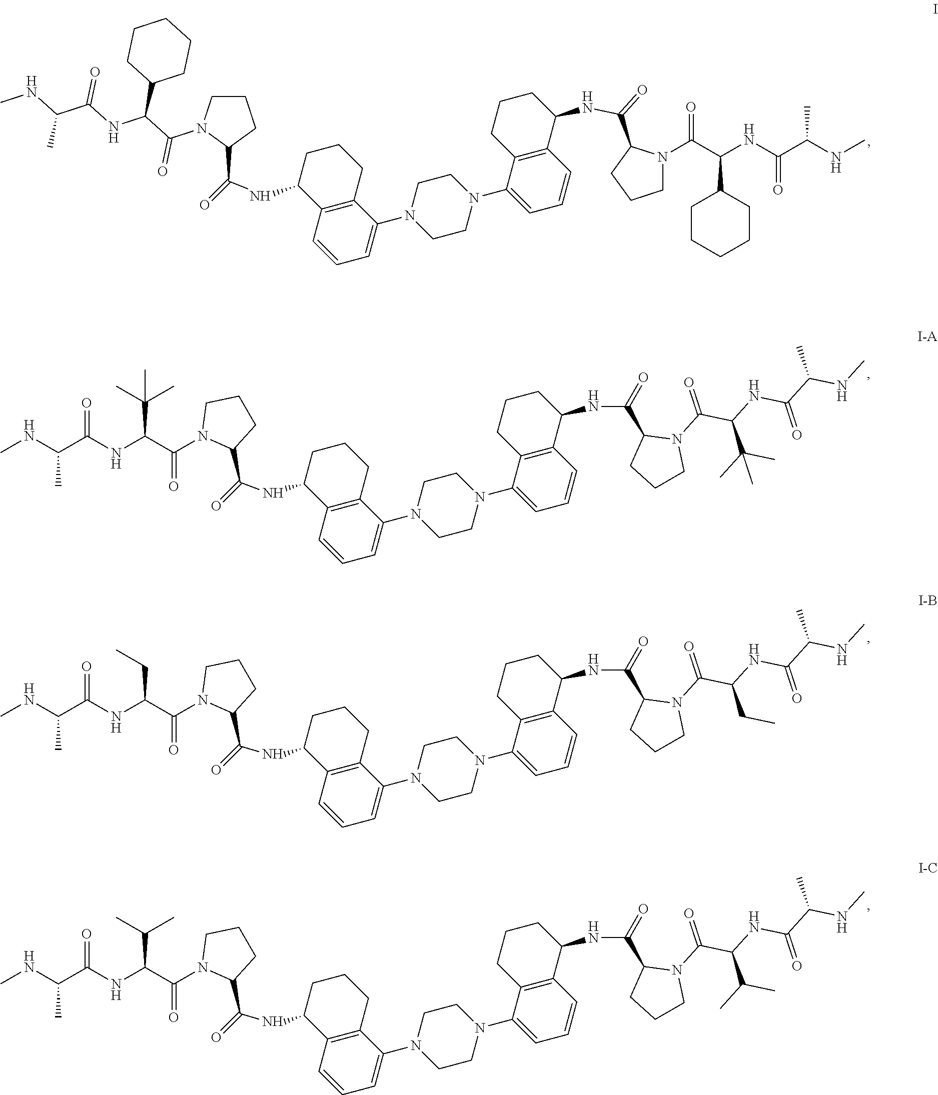

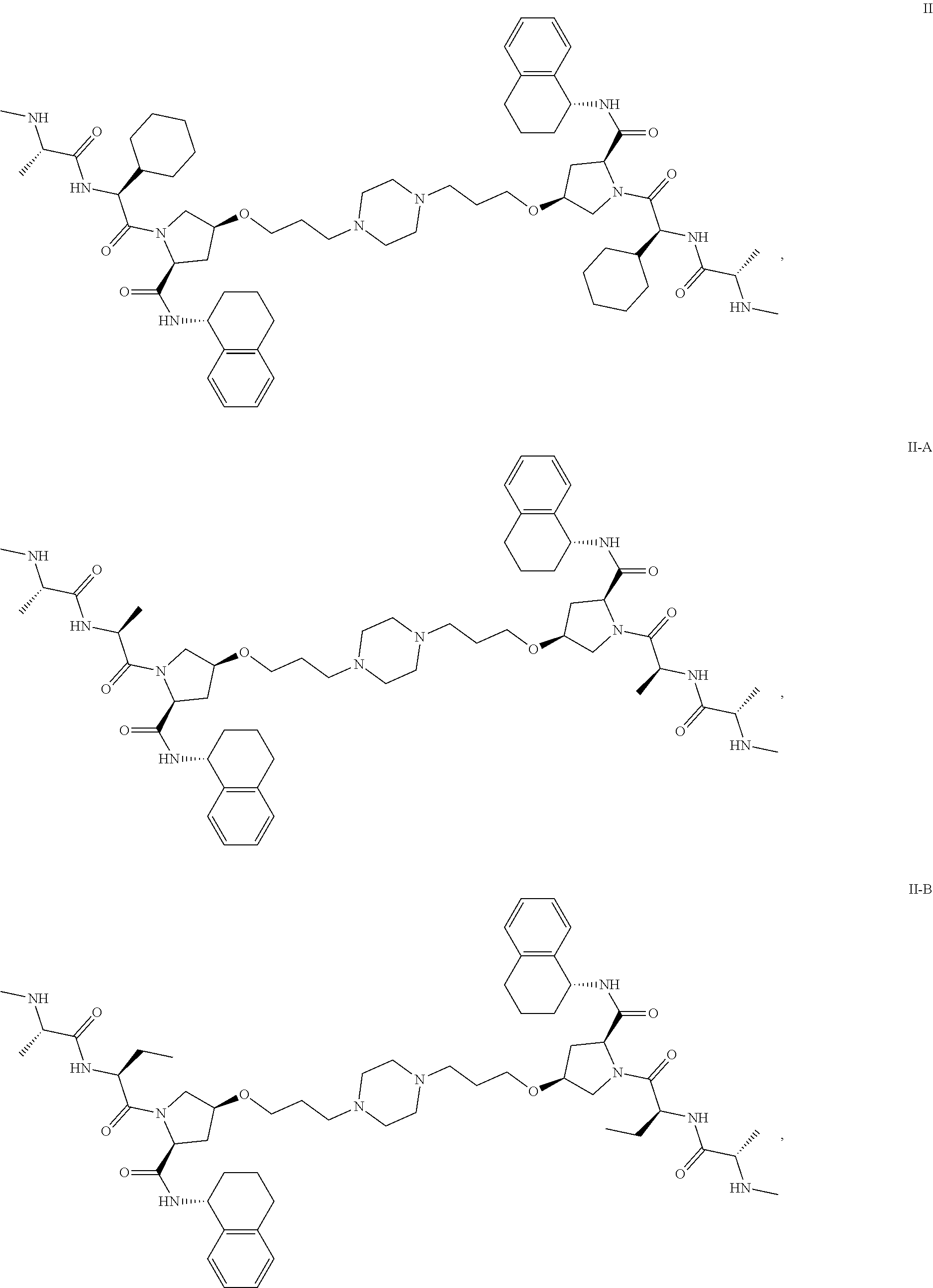

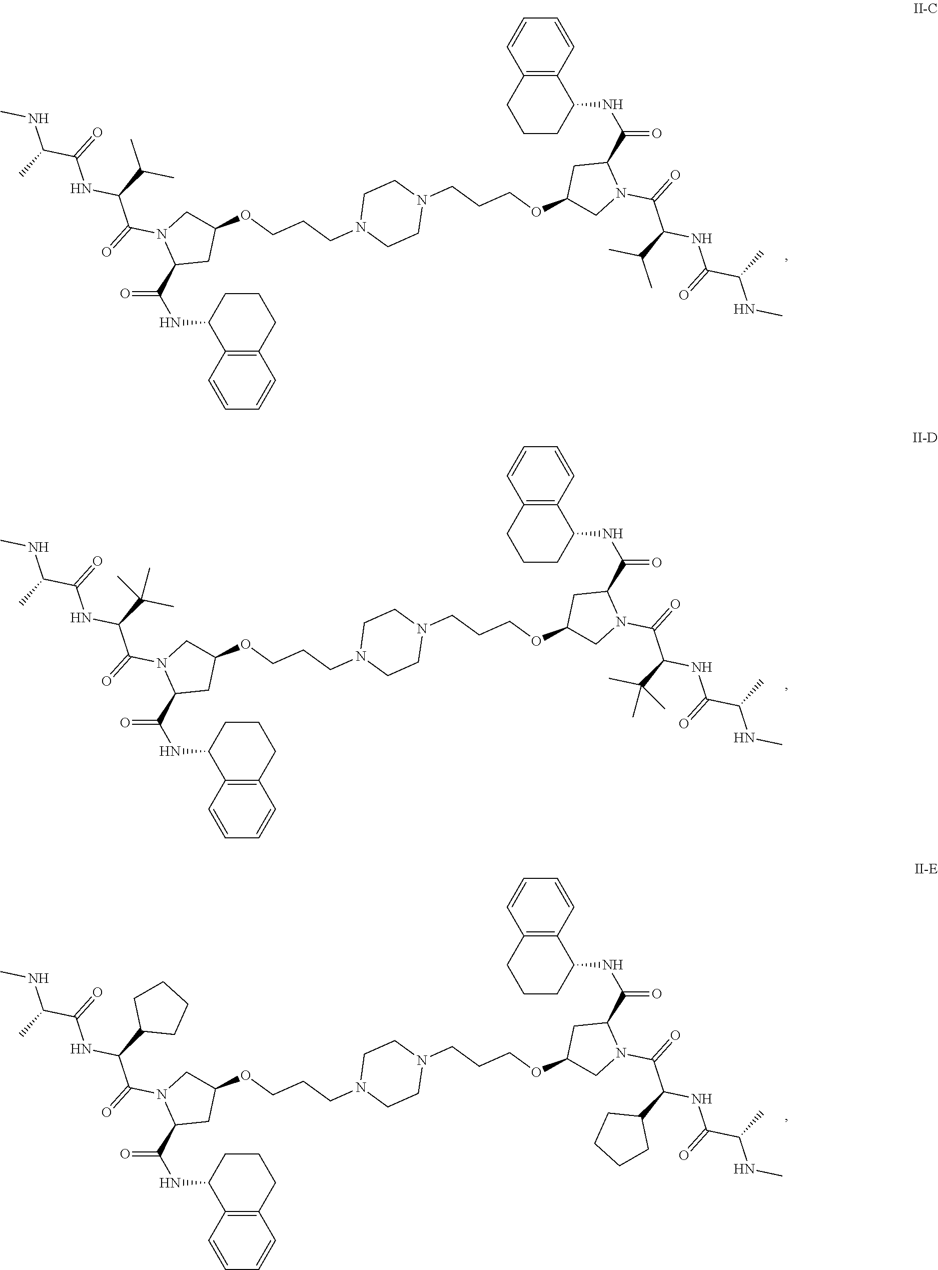

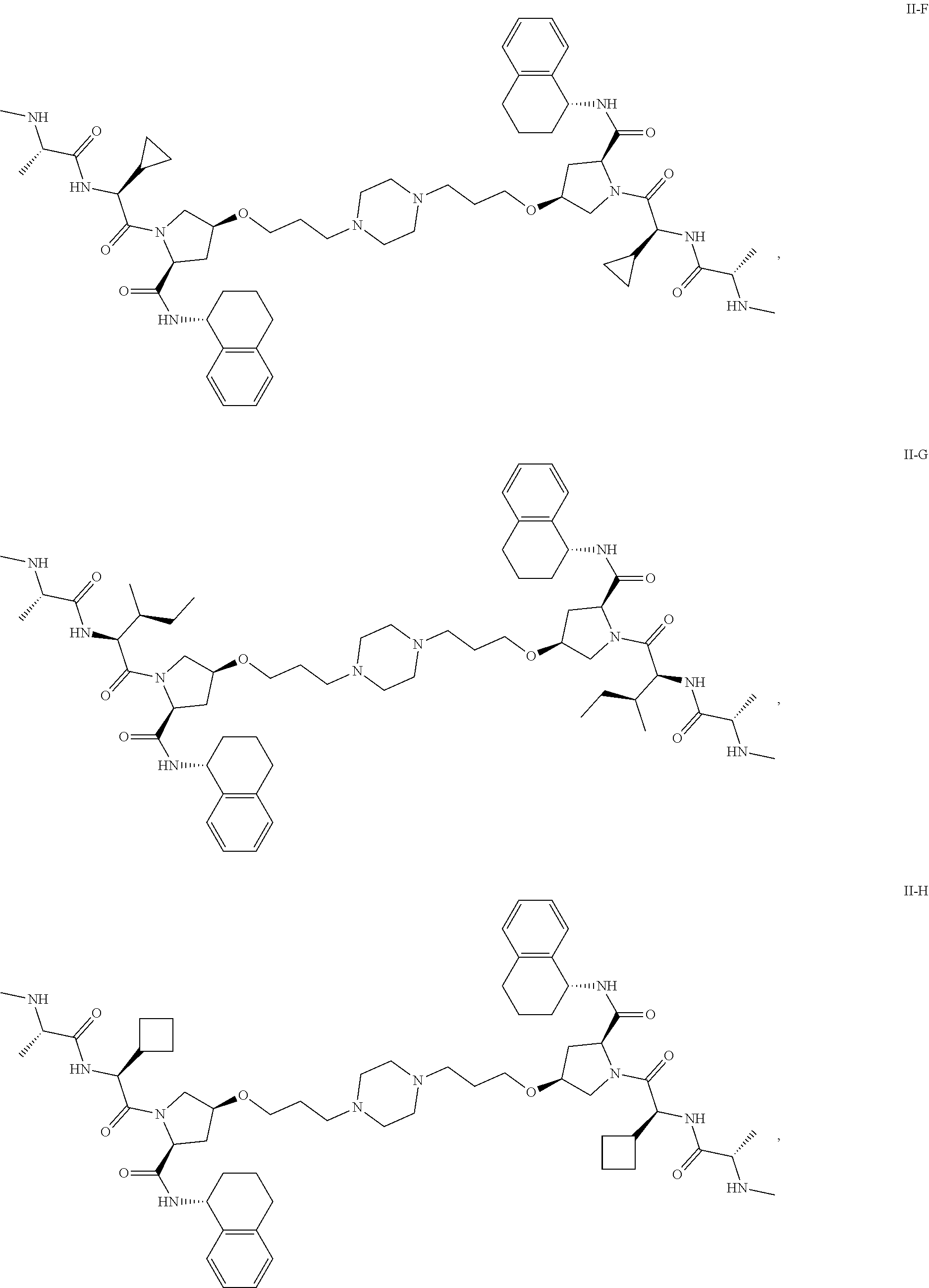

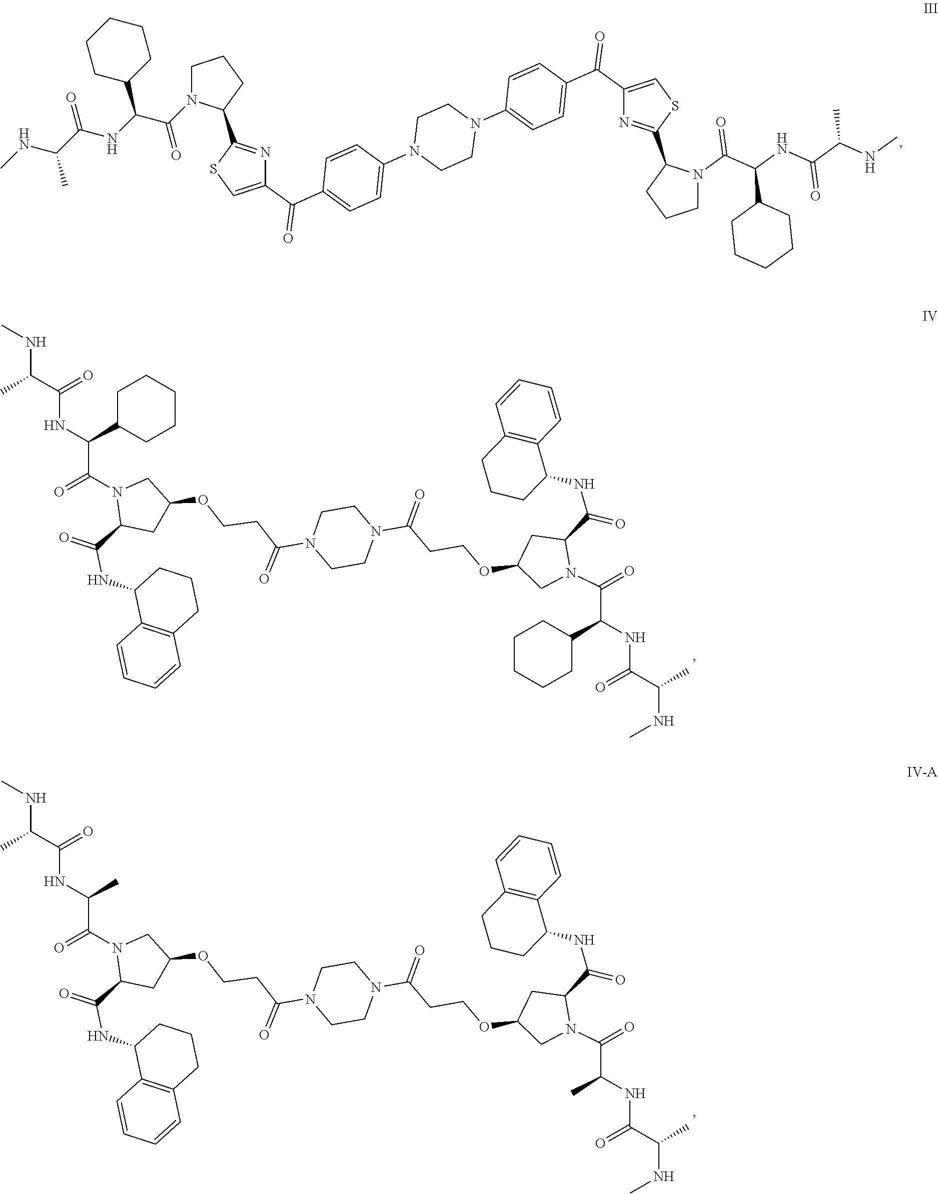

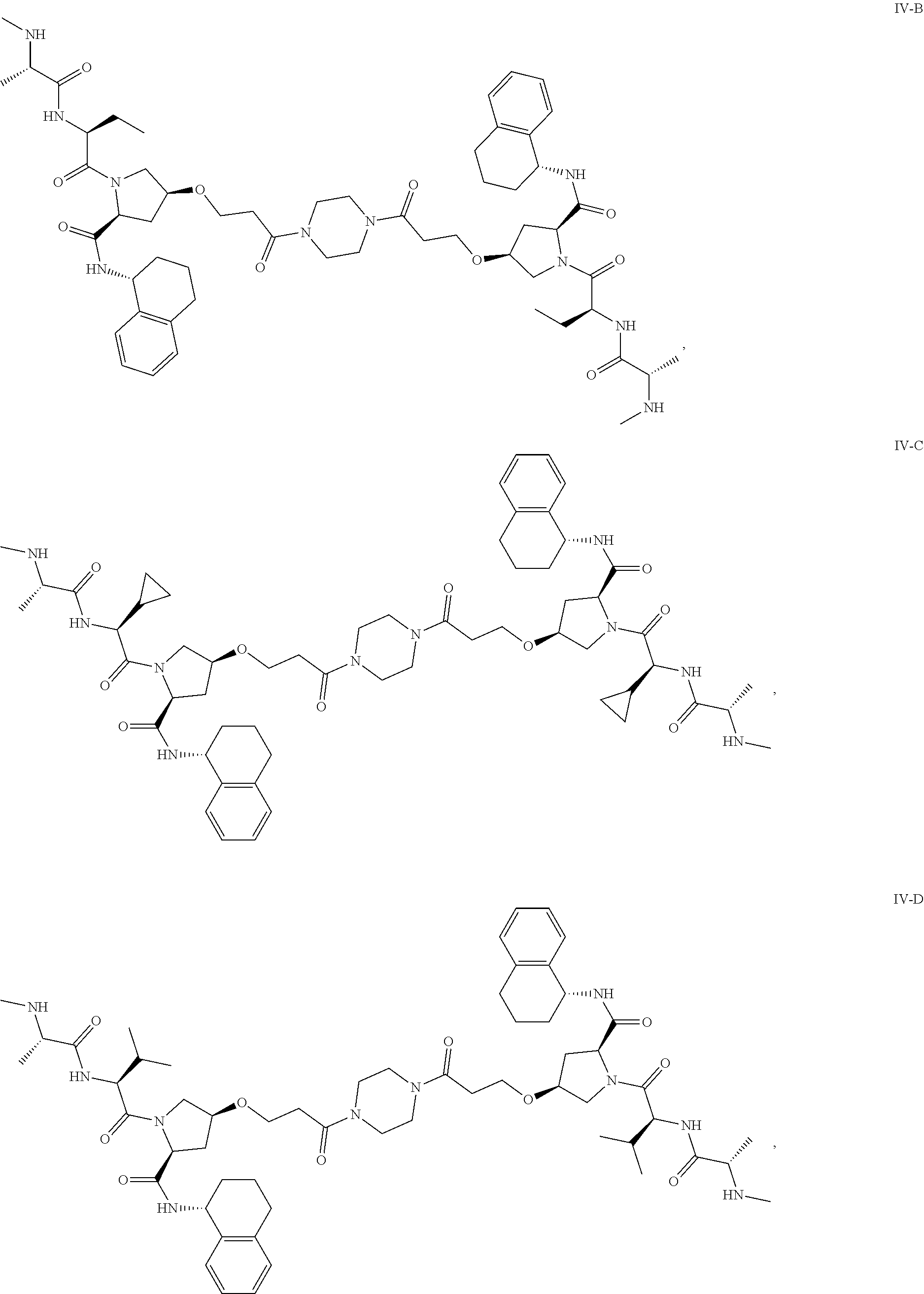

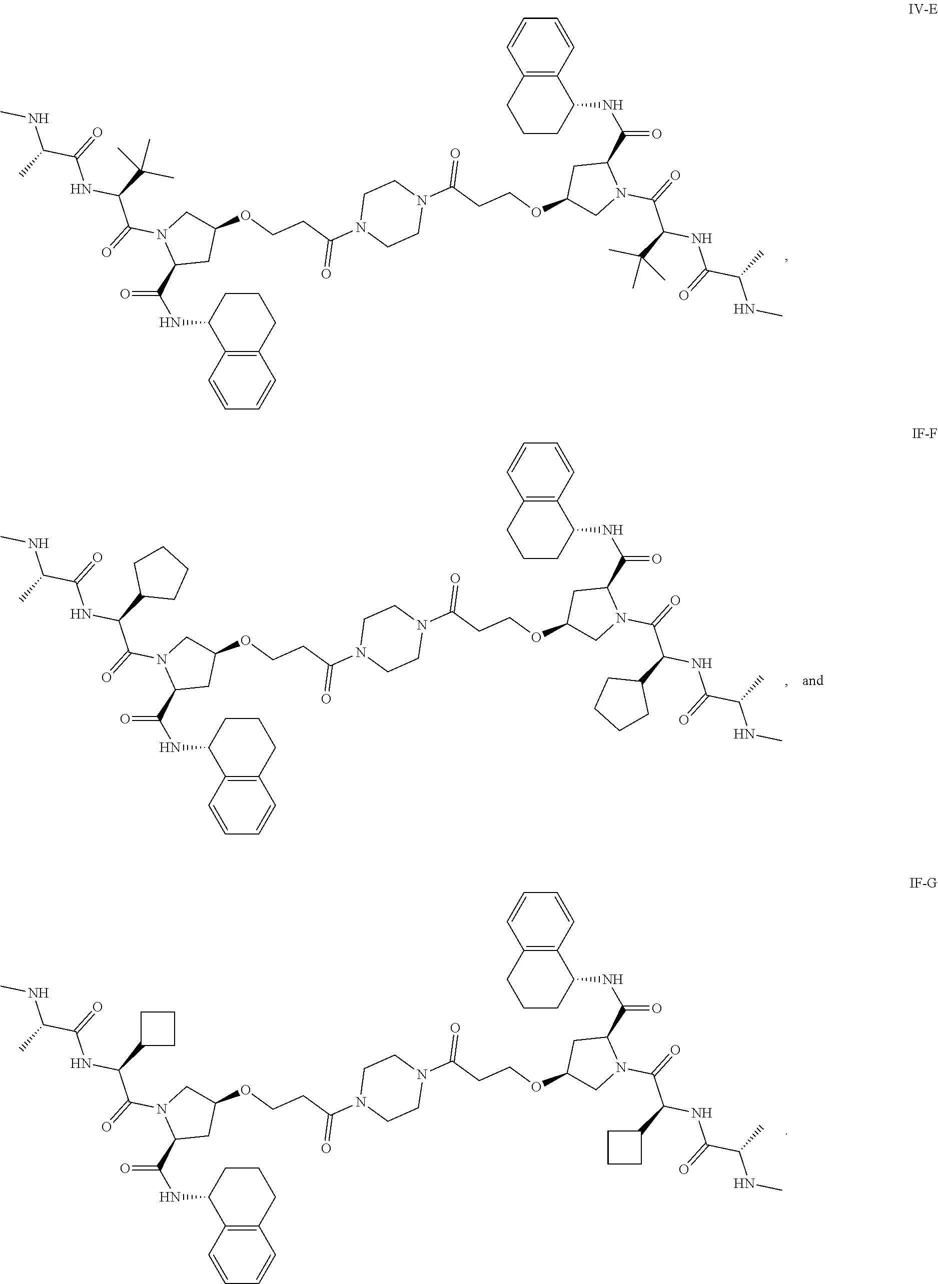

20. A composition comprising the compound of claim 1 and a pharmaceutically acceptable carrier.

21. A pharmaceutical composition comprising an effective amount of the compound of claim 1 for treating a cancer or a viral infection mediated by an IAP.

22. The pharmaceutical composition of claim 21 wherein the cancer or viral infection mediated by an IAP is selected from the group consisting of ovarian cancer, fallopian tube cancer, peritoneal cancer, and hepatitis B infection.

23. A method of treatment comprising administering an effective amount of a compound of claim 1, or administering a pharmaceutical composition comprising an effective amount of a compound of claim 1, to a subject suffering from a cancer or a viral infection mediated by an IAP.

24. The method of claim 23, wherein the cancer or viral infection is selected from the group consisting of ovarian cancer, fallopian tube cancer, peritoneal cancer, and hepatitis B infection.

25. A compound of Formula I, a stereoisomer thereof, or a pharmaceutically acceptable salt of the compound or the stereoisomer of the compound: 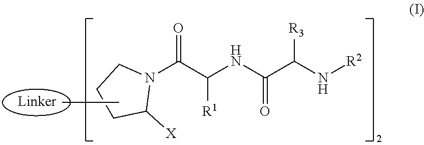
wherein
X is a bond to the Linker or, when the Linker is attached to positions, 2, 3, or 4 on the pyrrolidine ring, X is selected from 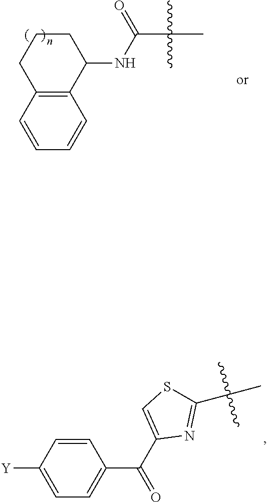
wherein
Y is H or halogen;
R¹ is a C₃₋₆ cycloalkyl group;
R² is H or a substituted or unsubstituted C₁₋₆ alkyl group;
R³ is selected from a substituted or unsubstituted C₁₋₆ alkyl or a C₃₋₆ cycloalkyl group;
m is 1, 2, 3, 4, 5, or 6;
n is 0, 1 or 2; and
Linker is

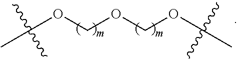

26. The compound of claim 25 wherein m is 2 or 3.

27. The compound of claim 25 the compound is

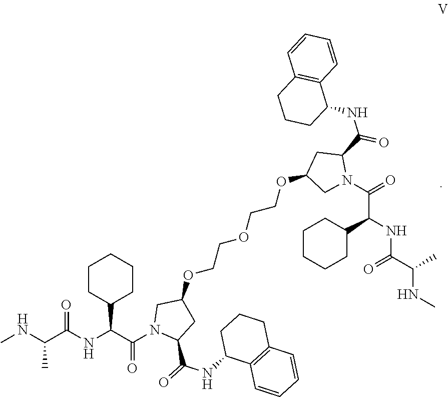 [SMILES: CN[C@@H](C)C(=O)N[C@H](C(=O)N1C[C@@H](OCCOCCO[C@H]2C[C@@H](C(=O)N[C@@H]3CCCc4ccccc43)N(C(=O)[C@@H](NC(=O)[C@H](C)NC)C3CCCCC3)C2)C[C@H]1C(=O)N[C@@H]1CCCc2ccccc21)C1CCCCC1]

28. A pharmaceutical composition comprising an effective amount of the compound of claim 25 for treating a cancer or a viral infection mediated by an IAP.

29. The pharmaceutical composition of claim 28 wherein the cancer or viral infection mediated by an IAP is selected from the group consisting of ovarian cancer, fallopian tube cancer, peritoneal cancer, and hepatitis B infection.

30. A method of treatment comprising administering an effective amount of a compound of claim 25, or administering a pharmaceutical composition comprising an effective amount of a compound of claim 25, to a subject suffering from a cancer or a viral infection mediated by an IAP.

31. The method of claim 30, wherein the cancer or viral infection is selected from the group consisting of ovarian cancer, fallopian tube cancer, peritoneal cancer, and hepatitis B infection.
